SEQUENCE LISTING (25 sequences)

SEQ ID NO: 1 - DNA, 21 bp, Homo sapiens
  source 1..21: mol_type = other DNA
cggctggtcctccaaacataa

SEQ ID NO: 2 - DNA, 20 bp, Homo sapiens
  source 1..20: mol_type = other DNA
cagaaccggaagagaggcaa

SEQ ID NO: 3 - DNA, 21 bp, Homo sapiens
  source 1..21: mol_type = other DNA
aactttcgatggtagtcgccg

SEQ ID NO: 4 - DNA, 21 bp, Homo sapiens
  source 1..21: mol_type = other DNA
ccttggatgtggtagccgttt

SEQ ID NO: 5 - DNA, 20 bp, Homo sapiens
  source 1..20: mol_type = other DNA
tacgccttctccaacagcag

SEQ ID NO: 6 - DNA, 20 bp, Homo sapiens
  source 1..20: mol_type = other DNA
gtcatgtgcatgaagccgtc

SEQ ID NO: 7 - DNA, 20 bp, Homo sapiens
  source 1..20: mol_type = other DNA
ggtgtggtcaacattgtgcc

SEQ ID NO: 8 - DNA, 20 bp, Homo sapiens
  source 1..20: mol_type = other DNA
attacgcggccaatctcagt

SEQ ID NO: 9 - DNA, 20 bp, Homo sapiens
  source 1..20: mol_type = other DNA
aagaggagatcttcgggcct

SEQ ID NO: 10 - DNA, 22 bp, Homo sapiens
  source 1..22: mol_type = other DNA
taatcaccttgtcgttgctgga

SEQ ID NO: 11 - DNA, 20 bp, Homo sapiens
  source 1..20: mol_type = other DNA
tcaacagggtcaccaaccag

SEQ ID NO: 12 - DNA, 21 bp, Homo sapiens
  source 1..21: mol_type = other DNA
ggggtctgggtatccttcaat

SEQ ID NO: 13 - DNA, 20 bp, Homo sapiens
  source 1..20: mol_type = other DNA
tggtggacctcatcgaacac

SEQ ID NO: 14 - DNA, 20 bp, Homo sapiens
  source 1..20: mol_type = other DNA
atgaagcctgggtcgtagtg

SEQ ID NO: 15 - DNA, 20 bp, Homo sapiens
  source 1..20: mol_type = other DNA
tgcgtgctgatcgtgatctt

SEQ ID NO: 16 - DNA, 25 bp, Homo sapiens
  source 1..25: mol_type = other DNA
cattcttggagtctttctaacgagc

SEQ ID NO: 17 - DNA, 20 bp, Homo sapiens
  source 1..20: mol_type = other DNA
cccagcaagaaggcgagtta

SEQ ID NO: 18 - DNA, 20 bp, Homo sapiens
  source 1..20: mol_type = other DNA
catcggccaaattcgtcacc

SEQ ID NO: 19 - DNA, 21 bp, Homo sapiens
  source 1..21: mol_type = other DNA
gccaaggaagctcactgccat

SEQ ID NO: 20 - DNA, 21 bp, Homo sapiens
  source 1..21: mol_type = other DNA
acggaaaggaacaggaagcat

SEQ ID NO: 21 - RNA, 20 nt, Homo sapiens
  source 1..20: mol_type = other RNA
ccgagtgcatcctgagcaag

SEQ ID NO: 22 - RNA, 20 nt, Homo sapiens
  source 1..20: mol_type = other RNA
gagtacctggtcaagtggcg

SEQ ID NO: 23 - DNA, 19 bp, Homo sapiens
  source 1..19: mol_type = other DNA
agcatggaggagctgagca

SEQ ID NO: 24 - DNA, 21 bp, Homo sapiens
  source 1..21: mol_type = other DNA
ggttacagcggggagaatctg

SEQ ID NO: 25 - DNA, 21 bp, Homo sapiens
  source 1..21: mol_type = other DNA
ggagaatctggccaagaggag

(19) United States
(12) Patent Application Publication
(10) Pub. No.: US 2025/0199004 A1
(43) Pub. Date: Jun. 19, 2025

(54) METHODS OF EVALUATING TREATMENT OUTCOME IN HIGH GRADE SEROUS OVARIAN CANCER

(71) Applicant: THE REGENTS OF THE UNIVERSITY OF COLORADO, A BODY CORPORATE, Denver, CO (US)

(72) Inventors: Benjamin G. Bitler, Denver, CO (US); Lindsay J. Wheeler, Denver, CO (US)

(21) Appl. No.: 19/043,313

(22) Filed: Jan. 31, 2025

Related U.S. Application Data
(63) Continuation-in-part of application No. 17/251,645, filed on Dec. 11, 2020.
(60) Provisional application No. 62/685,107, filed on Jun. 14, 2018.

(51) Int. Cl. G01N 33/574; G01N 33/53; G01N 33/573; G01N 33/68

(52) U.S. Cl. CPC G01N 33/574; G01N 33/5308; G01N 33/573; G01N 33/6893; G01N 2333/4703; G01N 2333/90203; G01N 2474/20

(57) ABSTRACT

Disclosed herein are method and compounds useful in the analysis, diagnosis, and treatment of high grade serous ovarian carcinoma (HGSOC). Also disclosed are methods, compounds, and compositions useful in regulating Chromobox 2 (CBX2) expression and therapies for stemness, anoikis escape, HGSOC dissemination, and HGSOC chemoresistance. Applicants have identified CBX2 expression as being significantly elevated in HGSOC cells and tissues compared to benign counterparts. Also disclosed is elevated CBX2 expression in HGSOC cell lines, as well as elevated CBX2 expression in cells that are forced to grown in suspension. CBX2 expression can be used to identify subjects for treatment with therapies specifically directed to cancers characterized by CBX2 upregulation.

### CROSS-REFERENCE TO RELATED APPLICATIONS

This is a continuation-in-part of U.S. patent application Ser. No. 17/251,645, filed on 11 Dec. 2020, entitled “Methods of Evaluating Treatment Outcome in High Grade Serous Ovarian Cancer,” which is a national stage application under 35 U.S.C. § 371 of International Application No. PCT/US2019/037289, filed on 14 Jun. 2019, entitled “Methods of Evaluating Treatment Outcome in High Grade Serous Ovarian Cancer,” which claims benefit of priority pursuant to 35 U.S.C. § 119(e) of U.S. provisional patent application No. 62/685,107 entitled “Methods of Evaluating Treatment Outcome in High Grade Serous Ovarian Cancer,” filed on 14 Jun. 2018, each which are hereby incorporated by reference in their entireties.

### GOVERNMENT LICENSE RIGHTS

This invention was made with government support under grant number CA194318 awarded by the National Institutes of Health (NIH). The government has certain rights in the invention.

### TECHNICAL FIELD

The disclosed processes, methods, and compounds are directed to diagnosing and treating conditions related to elevated CBX2 expression, especially in high-grade serous ovarian carcinoma cells and tissues.

### SEQUENCE LISTING

This application contains a Sequence Listing which has been submitted electronically in XML format via EFS-Web and is hereby incorporated by reference in its entirety. Said XML copy, created on Jan. 31, 2025, is named “P282460WOUS02_SequenceListing_ST26.xml” and is 23 kilobytes in size.

### BACKGROUND

Epithelial ovarian cancer is the deadliest gynecologic malignancy and annually accounts for over 220,000 deaths worldwide. In the US, over 22,000 new cases of ovarian cancer are diagnosed each year and over 14,000 women succumb to the disease. The majority of these cases are classified as high-grade serous ovarian carcinoma (HGSOC). HGSOC tends to be diagnosed at a late stage, when cancer has already spread beyond the pelvis, and will recur in the majority of cases. Current evidence suggests that HGSOC originates from transformed secretory fallopian tube epithelium (FTE) cells located on the fimbriated end of the fallopian tube. Precursor lesions, defined by TP53 mutations, include serous tubal intraepithelial carcinoma (STIC), which is focal and displays a cytologic appearance similar to HGSOC. Cells within STIC lesions demonstrate anoikis resistance or anchorage-independent cell survival by exfoliation from the fallopian tube-associated extracellular matrix and dissemination to the ovary and/or peritoneum. Ovarian, fallopian, and primary peritoneal carcinomas differ from other epithelial cancers that metastasize to distant sites predominantly via the circulatory or lymphatic systems (e.g., breast, endometrial) by spreading directly to the ovaries and the abdominal cavity independent of the lymphatic or vascular system. As HGSOC cells spread to the abdominal cavity they promote the production of ascites, a collection of intra-peritoneal fluid containing immune cells, tumor cells, and cytokines, along with other cellular and acellular factors. Notably, the prevalence of ascites is directly correlated to disease stage. For instance, 89% of stage III/IV patients present with some degree of ascites. Tumor cells within ascites are hypothesized to be a subpopulation of cells that contribute to disseminated, recurrent, and chemoresistant disease. However, the genetic drivers of HGSOC dissemination and anchorage-independent survival remain unclear.

A significant proportion of “stem”-like cells have been detected in the ascites fluid associated with HGSOC. One group of transcriptional repressors, the polycomb group (PcG) of proteins, are candidates for producing and maintaining this “stemness” as they have been shown to inhibit cellular differentiation and maintain a stem-like transcriptional program. PcG proteins assemble in two main Polycomb repressive complexes, PRC1 and PRC2. PRC1 and 2epigenetically repress pro-differentiation and tumor suppressor genes, and are important in several cancer types including prostate, breast, and HGSOC. Epigenetic “readers”, known as chromobox (CBX) proteins, play a critical role in PRC1 repressive activity by recognizing methylated histones through their chromobox domain. In 2014, Clermont et al. initially identified an oncogenic role for CBX2 through a genotranscriptomic meta-analysis in human cancers. In breast and prostate cancers, they reported that CBX2 upregulation and amplification significantly correlated with metastatic progression and lower overall survival. CBX2 depletion reduced cell viability and promoted apoptosis in metastatic prostate cancer, suggesting that CBX2 drives key regulators of cell proliferation and metastasis. Gui et al. evaluated the role of 12 PcG proteins in primary and recurrent ovarian cancer and found that immunohistochemistry (IHC) demonstrated significantly higher levels of CBX2 expression in recurrent tumors compared to primary tumors at presentation (primary ovarian tissue at presentation n=100, recurrent disease at relapse n=50, p<0.001). However, the role of CBX2 in HGSOC progression is unknown.

### SUMMARY

In one aspect, a method of classifying a tumor for treatment includes: obtaining a sample of a tumor; processing the sample to determine a measurement of Chromobox 2 (CBX2) expression in the tumor; assigning a score to the measurement; and classifying the tumor based on anticipated responsiveness to a treatment with an Aurora kinase A inhibitor, where classifying includes grading the score based upon a scale that includes a minimum score that identifies the tumor as likely responsive to the treatment. The method may also include where the measurement is an amount of CBX2 protein in the sample. The method may also include where processing includes performing an immunohistochemistry (IHC) assay on the sample, and where the score includes one or more of an H-score, Allred score, or immunoreactive score. The method may also include where the Aurora kinase A inhibitor is alisertib. Other technical features may be readily apparent to one skilled in the art from the following figures, descriptions, and claims. The method may also include where the score includes an H-score, and the minimum score is about 90 to about 300. The method may also include where the minimum score is at least about 120 or least about 150. The method may also include where the minimum score is at least two times greater than an H-score for an autologous non-tumor tissue sample. The method may also include where the minimum score is at least three times greater than an H-score for an autologous non-tumor tissue sample.

In one aspect, a method of determining a treatment for a patient with an ovarian cancer includes: obtaining a first sample of a tissue of Müllerian origin from the patient includes one or more cancerous or pre-cancerous cells; obtaining a second sample of a tissue of Müllerian origin from the patient that does not comprise cancerous or pre-cancerous cells; processing the first sample and the second sample to analyze at least one biomarker of Chromobox 2 (CBX2) expression, where the at least one biomarker includes one of: a CBX2 mRNA sequence, CBX2 protein or a fragment thereof, aldehyde dehydrogenase, and SOX4; and quantifying the amount of the at least one biomarker in the first sample and the second sample, where if the amount of the at least one biomarker in the first sample is greater than the amount of the at least one biomarker in the second sample, the patient is identified as having a cancer that is likely responsive to a treatment with an Aurora kinase inhibitor. The method may also include where the at least one biomarker is CBX2 protein or a fragment thereof, and where the quantifying step includes performing an immunohistochemistry (IHC) assay on the first sample and the second sample. The method may further include administering a therapeutically effective amount of the Aurora kinase A inhibitor to the patient. The method may also include where the Aurora kinase A inhibitor is alisertib. The method may further include assigning a score to each of the first sample and the second sample, where the score includes one or more of an H-score, Allred score, and an immunoreactive score. The method may also include where the score includes an H-score, and where the patient is identified as having a cancer that is likely responsive to a treatment with an Aurora kinase inhibitor if the H-score for the first sample is at least two times greater than the H-score for the second sample. The method may also include where the patient is so identified if the H-score for the first sample is at least three times greater than the H-score for the second sample.

In one aspect, a kit for classifying a tumor for treatment includes an article providing a scale of immunohistochemistry scores that indicates a minimum score identifying a sample having that score as coming from a tumor that is likely responsive to a treatment with an Aurora kinase inhibitor. The kit may also include where the article is a printed material. The kit may also include where the scale includes one or more of H-scores, Allred scores, and immunoreactive scores. The kit may also include where the scale includes H-scores, and the minimum score is about 90 to about 300.

Other technical features may be readily apparent to one skilled in the art from the following figures, descriptions, and claims.

### BRIEF DESCRIPTION OF THE DRAWINGS

FIG. 1A and FIG. 1B show CBX2 is overexpressed in high grade serous carcinoma and portends poor prognosis.

FIG. 2A and FIG. 2B show inhibition of CBX2 impairs HGSOC cell proliferation.

FIG. 3A and FIG. 3B show suspension growth and CBX2 modulation in multiple cell lines.

FIG. 4 shows CBX2 expressed in advanced HGSOC.

FIG. 5A and FIG. 5B show CBX2 antibody validation for immunohistochemistry.

FIG. 6 shows loss of CBX2 sensitizes HGSOC to chemotherapy.

FIG. 7A and FIG. 7B show CBX2 knockdown sensitizes OVCAR8 and PEO1 cells to cisplatin.

FIG. 8A and FIG. 8B show a correlation of CBX2 expression and autophagy, apoptosis, and EMT.

FIG. 9A and FIG. 9B show inhibition of CBX2 decreases stemness.

FIG. 10A and FIG. 10B show CBX2 knockdown leads to loss of ALDH3A1 expression.

FIG. 11 shows an evaluation of the interaction between alisertib and the function of CBX2 in PRC1.

FIG. 12 shows the impact of treatment with IC50 of top compounds on markers of CBX2 and PRC1.

FIG. 13 presents results showing that treatment with alisertib leads to decreased stem cell frequency.

FIG. 14 shows a further evaluation of the interaction between alisertib and the function of CBX2 in PRC1.

FIG. 15 shows an evaluation of the interaction between alisertib and the function of CBX2 in a syngeneic mouse model.

### DETAILED DESCRIPTION

Disclosed herein are studies showing that CBX2 is overexpressed in primary HGSOC tumors and that CBX2 protein is upregulated in HGSOC cells grown in an anchorage-independent fashion (forced suspension). In a primary human HGSOC tumor microarray high CBX2 expression was observed in a majority of specimens. Also shown is that the loss of CBX2 inhibits proliferation, reduces stemness, and increases cisplatin sensitivity. Also disclosed are methods of diagnosing advanced cases of HGSOC, HGSOC that may be resistant to one or more anti-cancer therapies, for example platinum-based chemotherapy, such as cisplatin therapy. Also disclosed are compounds and methods for treating a subject having advanced or chemoresistant HGSOC, wherein the compounds and methods reduce CBX2 expression in HGSOC cells and/or reduce dissemination of primary tumors.

High grade serous ovarian carcinoma (HGSOC) is often diagnosed at an advanced stage. Chromobox 2 (CBX2), a polycomb repressor complex subunit, plays an oncogenic role in other cancers, but little is known about its role in HGSOC. Applicants hypothesized that CBX2 upregulation promotes HGSOC via induction of a stem-like transcriptional profile and inhibition of anoikis. Examination of Gene Expression Omnibus (GEO) datasets and The Cancer Genome Atlas (TCGA) established that increased CBX2 expression conveyed chemoresistance and worse disease-free and overall survival. In primary HGSOC tumors, Applicants observed CBX2 expression was significantly elevated compared to benign counterparts. In HGSOC cell lines, forced suspension promoted CBX2 expression. Subsequently, CBX2 knockdown inhibited anchorage-independent proliferation and potentiated anoikis-dependent apoptosis. Furthermore, CBX2 knockdown re-sensitized cells to platinum-based chemotherapy. Forced suspension promoted increased ALDH activity and ALDH3A1 expression and CBX2 knockdown led to a decrease in both ALDH activity and ALDH3A1 expression. Investigation of CBX2 expression on a HGSOC tissue microarray revealed CBX2 expression was apparent in both primary and metastatic tissues. CBX2 is an important regulator of stemness, anoikis escape, HGSOC dissemination, and chemoresistance and potentially serves as a novel therapeutic target.

### Discussion

Applicants have discovered that CBX2 is upregulated in HGSOC, high CBX2 expression portends poorer survival, and increased CBX2 expression correlates with platinum resistance. Applicants have also discovered that CBX2 is overexpressed in HGSOC primary tumors, as well as in cell lines that have escaped anoikis in suspension culture. Here, Applicants demonstrate that the loss of CBX2 is associated with decreased proliferation of HGSOC cells in multiple culture conditions, an increase in chemotherapy sensitivity, and a reduction in stem-like cells. Lastly, utilizing a HGSOC tissue microarray of advanced stage primary patient tumors Applicants found CBX2 protein expression was expressed in a majority of tumors.

CBX2 was found to be highly expressed in primary HGSOC tumors from seven patients. Clinically, three of these patients had more extensive peritoneal disease (Table 1) suggesting that CBX2 could serve as a predictive marker of advanced disease, reinforcing the potential clinical significance of CBX2. Moreover, examination of five patients with matched primary tumor, ascites-associated tumor cells, and distant metastasis revealed three of the five patients had an increase in CBX2 expression in distant metastasis/ascites-associated tumor cells compared to primary tumors. This highlights that CBX2 is potentially important in driving HGSOC progression, however there are indeed other contributing factors. Notably we observed that high expression of CBX2 correlated to a loss of an active tumor suppressor, FOXO3. This suggests that the downregulation of FOXO3 independent of CBX2 could drive tumor progression. As we have attempted to elucidate the mechanism behind CBX2-dependent activity we see common themes of increased proliferation and stem-like differentiation, which appeared to lead to a more aggressive and chemoresistant phenotype. The loss of CBX2 led to the loss of stemness measured through ALDH activity, ALDH3A1 expression, and SOX4 expression suggesting that reduced stemness is a major driver of these phenotypes. In addition, we linked the anoikis-induced autophagy, EMT, and apoptosis response to CBX2 expression. Further investigations will functionally evaluate the relationship between CBX2 and these key survival processes.

TABLE 1
Clinical characteristics of patient-derived protein lysates

| Label | Specimen | Pathologic Diagnosis | Indication for surgery | Clinical diagnosis |
|---|---|---|---|---|
| FTE | Normal | HGSC, right fallopian | Left adnexal mass, left | Stage IIIC high |
| (952) | fallopian | tube not involved | hydroureter and | grade serous |
| | tube | | hydronephrosis | ovarian cancer, |
| | | | | recurrent |
| FTE | Normal | Endometrioid, | Endometrial | Stage IA |
| (968) | fallopian | endometrial | adenocarcinoma | Endometrioid, |
| | tube | adenocarcinoma, FIGO | diagnosed on biopsy | endometrial |
| | | grade 1 | | adenocarcinoma, |
| | | | | FIGO Grade I |
| FTE | Normal | Lipoleiomyomata, | Incidentally found 20 cm | Benign pelvic mass |
| (1008) | fallopian | bilateral fallopian tubes | right adnexal mass on CT | |
| | tube | without histopathologic | scan | |
| | | abnormality | | |
| Benign | Normal | Uterus, cervix, and | Malignant cells on pap | Negative for |
| (982) | ovary | bilateral ovaries and | smear | malignancy. |
| | | fallopian tubes negative | | |
| | | for malignancy | | |
| HGSC | Omentum | HGSC | Inflamed gallbladder, | Stage IVB high |
| (905) | | | omental cake, and | grade serous |
| | | | adnexal masses | ovarian carcinoma, |
| | | | suspicious for ovarian | recurrent |
| | | | cancer | |
| HGSC | Tumor/ | HGSC | Adnexal masses, | Stage IVB high |
| (930) | ovary | | concerning for ovarian | grade serous |
| | | | cancer | ovarian cancer, |
| | | | | recurrent |
| HGSC | Tumor/ | HGSC | Pelvic mass, ascites, and | Stage IIIC high |
| (945) | omentum/ | | carcinomatosis | grade serous |
| | ovary | | concerning for ovarian | ovarian cancer, |
| | | | cancer | recurrent |
| HGSC* | Tumor/ | HGSC | Abdominal distension, | Stage IIIC high |
| (966) | ovary | | bilateral adnexal masses | grade serous |
| | | | | ovarian cancer, |
| | | | | recurrent |
| HGSC | Tumor/ | HGSC | Omental caking and | Stage IIIC high |
| (995) | ovary | | adnexal masses | grade serous |
| | | | suspicious for ovarian | ovarian cancer |
| | | | cancer. | |
| HGSC | Tumor/ | HGSC | Left adnexal mass, left | Stage IIIC high |
| (952) | ovary | | hydroureter and | grade serous |
| | | | hydronephrosis | ovarian cancer, |
| | | | | recurrent |
| HGSC | Tumor/ | HGSC | Bilateral adnexal masses | Stage IIIC high |
| (1003) | ovary | | | grade serous |
| | | | | carcinoma of the |
| | | | | fallopian tube |
| Other | Tumor/ | Low grade serous | Flank pain, adnexal mass | Stage IV low grade |
| histotype | omentum | carcinoma | | serous ovarian |
| (953) | | | | cancer, recurrent |
| Other | Tumor/ | Carcinosarcoma | Pelvic mass in setting of | Stage IVB |
| histotype | ovary | | carcinoma of unknown | carcinosarcoma of |
| (950) | | | origin | the ovary |
| Other | Tumor/ | Carcinosarcoma | Small bowel obstruction, | Stage IIIC ovarian |
| histotype | ovary | | new diagnosis | carcinosarcoma |
| (991) | | | carcinosarcoma | |
| Other | Tumor/ | Mucinous | Ascites, bloating, | Stage IIIC |
| histotype | ovary | adenocarcinoma, | cytology positive for | mucinous |
| (993) | | intestinal type, primary | adenocarcinoma | adenocarcinoma of |
| | | tumor site: right ovary | | the ovary |

| Label | Surgical findings (when relevant) | Surgical outcome | CBX2 status |
|---|---|---|---|
| FTE | Bilateral adnexal masses 4 × 5 cm on the right and | Optimal | Faint |
| (952) | 8 × 10 cm on the left. Significant retroperitoneal | debulking | |
| | tumor burden, left greater than right necessitating | to <1 cm | |
| | aggressive dissection and ligation of internal iliac | residual | |
| | artery to remove tumor. | disease | |
| | Per pathology report: right fallopian tube NOT | | |
| | INVOLVED with HGSOC | | |
| FTE | Mildly enlarged uterus, normal-appearing right | Not | Faint |
| (968) | fallopian tube and ovary. Previous surgically | applicable. | |
| | absent left fallopian tube and ovary. There was no | | |
| | disease noted in the pelvis. | | |
| FTE | Enlarged, approximately 20 × 20 cm | Optimal | Faint |
| (1008) | retroperitoneal mass arising within the broad | debulking | |
| | ligament attached to the uterus. Both ovaries and | | |
| | fallopian tubes were normal bilaterally. Extensive | | |
| | retroperitoneal fibrosis, necessitating complete | | |
| | ureterolysis. | | |
| Benign | Uterus slightly enlarged and globular. Significant | Not applicable | Faint |
| (982) | intra-abdominal adhesions. *No specific mention | | |
| | of ovaries in findings | | |
| HGSC | Extensive approximately 30 × 20 × 10 cm omental | Optimal | Bright |
| (905) | cake incorporating the infracolic and gastrocolic | debulking | |
| | omentum, removed completely. Splenic hilar | to <1 cm, | |
| | tumor and distal pancreatic surface tumor, | however | |
| | removed completely with splenectomy and distal | residual | |
| | pancreatectomy. Diffuse intraabdominal | consisted of | |
| | mesenteric and peritoneal masses up to 4 cm in | thousands of | |
| | size, debulked with the use of argon beam to less | less than 1 cm | |
| | than 1 cm thickness of the removed plaques. Right | diffuse | |
| | adnexal mass approximately 8 cm. Diffuse | peritoneal and | |
| | peritoneal disease measuring in the thousands, all | mesenteric | |
| | debulked to less than 1 cm, discrete lesions or | nodules, as | |
| | plaque-like in thickness areas. Gallbladder | well as areas | |
| | removed with thickened wall of the gallbladder, | of plaque | |
| | but no intrinsic luminal tumor. | which had | |
| | | been debulked | |
| | | to less than 1 | |
| | | cm thickness, | |
| | | but | |
| | | nonetheless | |
| | | consisted of | |
| | | plaque-like | |
| | | disease. | |
| HGSC | Bilateral adnexal masses, left with surface | Poor candidate | Bright |
| (930) | involvement with a tumor process, approximately | for primary | |
| | 10 cm in size. The right approximately 15 cm in | debulking | |
| | size and mostly cystic. Small volume | given | |
| | intraabdominal ascites, approximately 50 cc. The | extensive right | |
| | right hemidiaphragm infiltration approximately 3 | diaphragmatic | |
| | to 4 cm thick and across an approximately 10 cm | disease. | |
| | area making it difficult to remove without | Underwent | |
| | complete diaphragmatic resection. No obviously | optimal | |
| | thickened omentum on laparoscopic assessment. | cytoreduction | |
| | Multiple small nodules ranging from 5 to 10 mm | to R0 after 3 | |
| | diffusely through the peritoneum. | cycles of | |
| | | neoadjuvant | |
| | | chemotherapy | |
| HGSC | Approximately 2 L of ascites. Omental disease | Optimal | Moderate |
| (945) | with numerous peritoneal-based lesions, the | debulking | |
| | majority less than 1 cm and those greater than 1 | to <1 cm, | |
| | cm debulked to less than 1 cm residual volume. | however | |
| | Tumor roll in the omentum approximately 15 cm × | innumerable | |
| | 9 cm and 4 cm, debulked to less than 1 cm | miliary disease | |
| | residual. Bilateral adnexal masses, approximately | on the bilateral | |
| | 10 cm on the left and approximately 4 cm on the | diaphragms | |
| | right, densely adherent in the pelvis, with bilateral | and peritoneal | |
| | ureterolysis secondary to retroperitoneal fibrosis | surfaces | |
| | allowing resection of the ovaries. Anterior roll of | | |
| | tumor in the bladder peritoneum approximately | | |
| | 2 × 4 × 2 cm, completely removed. Tumor along the | | |
| | cecum and appendix resulting in removal of what | | |
| | appeared to be appendix involved with tumor, | | |
| | approximately 2 cm × 2 cm × 4 cm. Tumor mass | | |
| | in the lesser curvature of the stomach | | |
| | approximately 2 × 4 × 2 cm, removed completely. | | |
| | At the completion of the procedure, all disease | | |
| | was less than 1 cm, but did involve innumerable | | |
| | miliary disease on the subdiaphragms, as well as | | |
| | throughout all peritoneal surfaces. | | |
| HGSC* | Bilateral adnexal masses the right was | Optimal | Moderate |
| (966) | approximately 10 to 12 cm, left approximately 6 | primary | |
| | cm. There were multiple small nodules within the | debulking | |
| | omentum that appeared to be tumor. There was an | to R0 | |
| | approximately 2 cm nodule on the cecum that was | | |
| | sitting outside of the pelvis. There was a | | |
| | centimeter nodule sitting on the lower rectum in | | |
| | the pelvis, multiple subcentimeter nodules located | | |
| | in the lower abdomen, and the diaphragm on the | | |
| | right side was thinly coated with tumor that was | | |
| | ablated and bluntly dissected off with the Argon | | |
| | beam coagulation, so that this patient was | | |
| | optimally visually debulked to R0 disease. Pelvic | | |
| | and periaortic lymph node sampling was | | |
| | performed, for what appeared to be bulky disease | | |
| | in the periaortic lymph node bed. | | |
| HGSC | Omental mass 20 × 10 × 5 cm thick removed | Optimal | moderate |
| (995) | completely with omentectomy, bilateral adnexal | debulking | |
| | masses approximately 6 × 7 cm bilaterally, | to <0.5 cm | |
| | removed completely. Uterus slightly enlarged with | | |
| | an estimated weight of approximately 250 grams, | | |
| | removed completely, posterior carpets of | | |
| | peritoneal disease removed completely, with | | |
| | posterior cul-de-sac peritoneal stripping with | | |
| | argon beam to remove residual tumor in the pelvis | | |
| | down to less than 5 mm of residual disease. | | |
| | Bilateral retroperitoneal fibrosis necessitating | | |
| | dissection of the perirectal and perivesical spaces, | | |
| | and bilateral ureterolysis to allow complete | | |
| | resection of tumor and uterus secondary to the | | |
| | extensive dissection and the posterior cul-de-sac | | |
| | stripping. Diffuse 1 to 2 cm peritoneal disease | | |
| | debulked with excision or argon beam to less than | | |
| | 5 mm of residual, 5 mm thick, approximately 1 cm | | |
| | wide, scarred area at the dome of the liver in close | | |
| | approximation to the inferior vena cava which, | | |
| | was left in situ. | | |
| HGSC | Bilateral adnexal masses 4 × 5 cm on the right and | Optimal | Bright |
| (952) | 8 × 10 cm on the left. Significant retroperitoneal | debulking | |
| | tumor burden, left greater than right necessitating | to <1 cm | |
| | aggressive dissection and ligation of internal iliac | residual | |
| | artery to remove tumor. | disease | |
| HGSC | Extensive omental caking and adhered pelvis | Optimal | Faint- |
| (1003) | likely due to tumor with obliteration of the | debulking | moderate |
| | posterior cul-de-sac and the anterior cul-de-sac. | to <0.5 cm | |
| | Minimal normal tissue was able to be identified. | | |
| | Diaphragmatic studding and extensive miliary | | |
| | tumor disease along the mesentery of the small | | |
| | and large bowel. Otherwise, no obvious large | | |
| | tumor nodules were noted, and there was no | | |
| | lymphadenopathy. | | |
| Other | Bilateral adnexal masses as described on CT scan, | Optimal | Faint |
| histotype | with omental masses adherent to the uterus and | primary | |
| (953) | the adnexal masses completely resected. The | debulking | |
| | omentum had multiple areas of confluent tumor 3 | to R0 | |
| | to 4 × 4 cm in diameter, as well as multiple other | | |
| | peritoneal based lesions measuring anywhere | | |
| | between 1 to 4 cm and numbering approximately | | |
| | 100 or so. These were all completely removed | | |
| | and/or debulked with the use of the argon beam | | |
| | coagulator. There was also additional upper | | |
| | abdominal disease and splenic lesions completely | | |
| | resected by the team by consulting service. | | |
| | Debulking of the diaphragmatic lesion involved | | |
| | resection of full-thickness diaphragm, and the | | |
| | chest tube was inserted intraoperatively. | | |
| Other | Enlarged, approximately 8 to 10 cm, multicystic | Delayed | Negative |
| histotype | and solid complex right adnexal mass that was | optimal | |
| (950) | densely adherent to the bladder, as well as an | primary | |
| | enlarged complex adnexal mass of the left ovary | debulking | |
| | that was densely adherent to the left pelvic | to R0 | |
| | sidewall. Pelvic and periaortic lymph node beds | | |
| | were palpated, with no adenopathy demonstrated. | | |
| | Omentum had minimal tissue and fat, with no | | |
| | visible disease. The liver edges felt smooth, | | |
| | without evidence of disease bilaterally. There was | | |
| | an approximately 4 cm to 5 cm right | | |
| | diaphragmatic lesion that was removed by | | |
| | consulting surgeon. There was no other palpable | | |
| | or visible disease throughout the abdomen or | | |
| | pelvis. | | |
| Other | Omental cake, approximately 7 × 8 cm × | Optimal | Negative |
| histotype | approximately 4 cm thick, removed completely. | debulking | |
| (991) | Right adnexal mass, approximately 4 × 5 cm, with | to 1-2 mm | |
| | adhesions of the terminal ileum and cecum to the | | |
| | area of the mass causing upstream bowel | | |
| | obstruction, removed completely and debulked | | |
| | with Argon beam coagulator to less than 1 to 2 | | |
| | mm residual. Plaque of tumor in the deep pelvis | | |
| | reduced to approximately 1 to 2 mm plaque | | |
| | residual with the use of Argon beam coagulator. | | |
| | No clearly identifiable left ovary and tube; hence, | | |
| | the region of the left ovary in between the IP | | |
| | ligament above the ureter and to the pelvic | | |
| | sidewall was removed and sent together as left | | |
| | ovary and tube. | | |
| Other | Darksih brown ascites drained, approximately 9 | Optimal tumor | Faint |
| histotype | liters. Enlarged bilateral adnexal masses. Uterus | debulking | |
| (993) | was small; however, there was prior disease to the | | |
| | anterior cul-de-sac, and the bladder posteriorly in | | |
| | the cul-de-sac. There was evidence of some | | |
| | inflammation, as well as possible previous disease | | |
| | that was treated by chemotherapy. There was then | | |
| | an approximate 2 cm nodule on the omentum that | | |
| | was taken out with the omentectomy. There were | | |
| | also adhesions and thickening on the anterior | | |
| | abdominal wall. Liver felt completely smooth. | | |
| | Peritoneum inflamed. Small bowel and large | | |
| | bowel grossly normal. Appendix grossly normal. | | |

CBX2 is a subunit of the polycomb repressor complex (PRC1), which has been shown to play a role in ovarian cancer. The enzymatic subunit or “writer” of PRC1, BMI-1, is considered to play a role in malignant transformation of multiple cancers, including ovarian cancer. In ovarian cancer, BMI-1 has been demonstrated to be associated with stemness and tumor initiation and serves as an independent predictor of poor outcome. Moreover, silencing of BMI-1 can lead to improved sensitivity to chemotherapy. This understanding of BMI-1 directly correlates and aligns with our CBX2 findings. Taken together with observations in other types of cancer, it seems likely that the PRC1 and specifically, CBX2, are novel therapeutic targets not only for HGSOC, but potentially for breast and prostate cancers.

CBX2 is considered to be an epigenetic “reader”. The existing literature suggests that targeting epigenetic “readers” is an effective strategy for targeted therapy. Bromodomain (acetyl-histone “reader”) inhibitors have the potential to suppress ALDH activity in ovarian cancer, providing evidence that targeting of an epigenetic reader may be able to alter the stem-like phenotype of a cell. One key example is JQ-1, a potent and selective inhibitor of the bromodomain and extra-terminal domain (BET) family of proteins, including BRD2, BRD3, and BRD4. Preliminary clinical data suggests that BET inhibitors may have therapeutic potential in human cancers. Furthermore, a Phase I clinical trial of an oral BET inhibitor demonstrated that this small molecule inhibitor was well tolerated in vivo, a critical development which paves the way for future targeting of epigenetic readers. This highlights that a “reader” or chromobox domain inhibitor could prove to be more effective with fewer adverse effects compared to inhibitors of epigenetic “writer” enzymes.

Disclosed herein, Applicants describe the role of CBX2 in promoting HGSOC disease progression. Mechanistically, CBX2 protects HGSOC against apoptosis and promotes a more stem-like phenotype. CBX2 is an epigenetic reader and is therefore targetable with a small molecule inhibitor. This work expands our understanding of the progression of HGSOC and identifies a novel therapeutic target.

Increased CBX2 protein in HGSOC (compared to fallopian tube epithelium, and other histotypes) is associated with poorer prognosis for the patient, repression of the FOXO3 tumor suppressor, and chemoresistance of HGSOC tumors and cells. CBX2 expression may be determined by various methods. In some embodiments, CBX2 protein expression may be assessed by densitometry of western immunoblot study. In many embodiments, increased expression may refer to CBX2 intensity that is greater than about 10%, 20%, 30%, 40%, 50%, 60%, 70%, 80%, 90%, 100%, 110%, 120%, 130%, 140%, 150%, 200%, 300%, 400%, 500%, 600%, 700%, 800%, or 900%, and less than about 1000%, 900%, 800%, 700%, 600%, 500%, 400%, 300%, 200%, 160%, 150%, 140%, 130%, 120%, 110%, 100%, 90%, 80%, 70%, 60%, 50%, 40%, 30%, or 20% compared to CBX2 in non-HGSOC cells. In other embodiments, relative CBX2 protein expression may be determined by other methods for quantitation of protein expression that are well-known to those of skill in the art.

In many embodiments, CBX2 protein may be identified by an antibody specific to CBX2, for example anti-CBX2 antibody from Thermo Fisher Scientific (Cat #PA5-30996). Relative quantitation of CBX2 may be adjusted or standardized by comparing CBX2 amounts to various control proteins, for example actin. CBX2 expression may be quantified by various methods. In some embodiments, CBX2 protein is analyzed and/or quantified by flow cytometry or mass spectrometry.

In some embodiments CBX2 protein expression can be quantified by performing IHC on a sample e.g., from a tumor. An example is an IHC assay that measures the amount of the CBX2 protein in a tissue section of a tumor sample by using an antibody or antigen-binding fragment thereof that binds to CBX2. Processing, or developing, an assay for visualization of antibody binding and measurement of CBX2 level can be accomplished by a variety of methods known in the art. Development and quantification of antibody binding to CBX2 can be through a detectable label, where labeling can be direct or indirect. The label can be colorimetric, fluorescent or radioactive. In some variations, an antibody which binds to CBX2 may directly comprise a detectable label. In other variations, development can utilize a reagent, such as a secondary antibody or biotin-avidin complex, that comprises a label and binds to the CBX2-binding antibody.

Reducing CBX2 expression, for example by various knockdown or repression methods, significantly decreases viability of HGSOC cells. In many embodiments, repression or knockdown leads to decreased growth rate and/or anoikis (anchorage-independent cell death) in HGSOC cells.

Suppression of CBX2 expression can promote cell death in HGSOC cells and/or reduce their growth rate. Suppression may be accomplished by various methods. In some embodiments, CBX2 mRNA transcripts may be targeted by one or more compounds including, but not limited to, siRNA, ribozyme, antisense, aptamer, or other small molecule. In some embodiments, CBX2 repression or knockdown may be accomplished by targeting CBX2 protein with one or more of, but not limited to, an antibody, peptide, peptidomimetic, small molecule, or other compound. In one embodiment, CBX2 transcripts may be targeted by one or more short hairpin RNA (shRNA) molecules, or other types of RNAi well-known to those of skill in the art. In some embodiments, the shRNA molecules may comprise a sequence selected from GCCAAGGAAGCTCACTGCCAT (shCBX2 #1; SEQ ID NO:19) or ACGGAAAGGAACAGGAAGCAT (shCBX2 #2; SEQ ID NO:20). In many embodiments, HGSOC cells may be induced to reduce their growth rate or enter anoikis by reducing the amount of CBX2 transcripts by greater than about 5%, 10%, 20%, 30%, 40%, 50%, 60%, 70%, 80%, 90%, or 95%, and less than about 100%, 90%, 80%, 70%, 60%, 50%, 40%, 30%, 20%, or 10% compared to HGSOC cells that have not been treated to reduce their CBX2 expression.

Patients having HGSOC cells with enhanced expression of CBX2 indicates that chemotherapy may be less effective. In some embodiments, the chemotherapy is a platinum-based compound, for example selected from one or more of cisplatin, carboplatin, and oxaplatin. In some embodiments, patients whose HGSOC tumor cells express high levels of CBX2 require an increased dosage of chemotherapeutic drug of greater than 10%, 20%, 30%, 40%, 50%, 60%, 70%, 80%, 90%, 100%, 150%, 200%, 250%, 300%, or 400%, and less than about 500%, 400%, 300%, 200%, 1500%, 100%, 90%, 80%, 70%, 60%, 50%, 40%, 30%, or 20%, compared to patients with tumors having lower CBX2 expression.

Repression of CBX2 expression can increase sensitivity of HGSOC cells to chemotherapy. In some embodiments, various therapies may be used to reduce expression of CBX2 in HGSOC cells and tumors. This may allow the use of various chemotherapeutic compounds to treat a patient positive for HGSOC and/or may allow the use of lower doses of chemotherapy to achieve a beneficial therapeutic effect, such as a reduction in tumor growth, lower cell growth, an increase in cancer cell death, or reduction in adverse side effects such as fatigue, hair loss, bruising, bleeding; infection; low red blood cell counts; nausea, etc.

CBX2 expression correlates to mRNA expression of genes associated with apoptosis, autophagy, and epithelial to mesenchymal transition (EMT). In many embodiments, the genes may include one or more of MYLK, NOG, and TNFSF10.

Increased CBX expression results in driving cells toward a stem-like phenotype. In many embodiments, a stem-like phenotype may be evaluated by measuring aldehyde dehydrogenase (ALDH), where stemness is associated with increased ALDH activity. In many embodiments, CBX2 expression correlates with ALDH3A1 expression, and knockdown of CBX2 expression decreases ALDH3A1 expression, as well as the stem cell-associated transcription factor, SOX4.

It is postulated that Aurora kinase inhibitors may provide a therapeutic effect against certain cancers that are characterized by CBX2 upregulation, such as HGSOC. For example, the Applicants have discovered indications of binding and/or other interaction between a small-molecule Aurora kinase A inhibitor and CBX2. Particularly, the Applicants have confirmed the functional dependence of the inhibitor on intact CBX2, where knockout of CBX2 was observed to attenuate the compound's effect. Additionally, a surrogate marker of broader PRC1 activity, H2AK119ub, was seen to decrease in parallel with CBX2 after treatment with the inhibitor.

The present disclosure provides various embodiments of a method of determining a treatment for a patient with a cancerous tumor, based on identifying the tumor/patient as being likely to respond to treatment with an Aurora kinase A inhibitor. In some embodiments, the determination can be made upon measuring an elevated level CBX2 expression in the patient's tumor sample. In some embodiments, the determination can be made by comparing levels of CBX2 expression in cancerous and non-cancerous tissue samples from the patient. As described above, CBX2 expression level may be ascertained by measuring a level of CBX2 protein or a fragment thereof. Alternatively, or in addition thereto, transcription al biomarkers such as CBX2 mRNA or downstream expression products such as ALDH or SOX4 may be measured to ascertain CBX2 expression level.

In some embodiments, these methods can comprise determining a measurement of CBX2 expression in a sample, and then assigning to the measurement a score that can be used to classify or otherwise identify the sample based on anticipated responsiveness to treatment with an Aurora kinase A inhibitor. This can involve grading the score based upon a scale of such scores. The scale can indicate a minimum score that identifies the tumor as likely responsive to the treatment.

In some embodiments, CBX2 expression levels can be assessed by performing an IHC assay on a sample, and assigning a score to an output of the assay. Particularly the score can comprise an IHC score such as one or more of an H-score, an Allred score, or an immunoreactive score (IRS).

As used herein, the term “H-score” is used to mean an immunohistology score for a tumor sample, in which the degree of IHC staining, if any, in each sub-cellular compartment in sample cells is captured for each analyte. This algorithm includes capturing the percentage of cells stained at each intensity level. A semi-quantitative intensity scale ranging from 0 for no staining to 3+for the most intense staining is used. This information can be used to calculate a continuous variable, the H-Score. An H-Score is typically calculated for staining of each sub-cellular compartment for both sample cells using the following formula; H-Score=(% cells at 0)*0+(% cells at 1+)*1+(% cells at 2+)*2+(% cells at 3+)*3. Thus, this score produces a continuous variable that ranges from 0 to 300.

In some embodiments, when the H-score is higher than a minimum or “cut-off” score, anti-tumor effects may be provided by chemotherapy that comprises an Aurora kinase A inhibitor. For example, when the subject has an H-score of about 90 to about 300, or more particularly greater than about 120 or about 150, the subject may be identified as responsive to treatment with an Aurora kinase A inhibitor. In some embodiments, the immunohistology score may be compared to an autologous sample of non-cancerous tissue. For example, the minimum score identifying a subject as responsive to treatment with an Aurora kinase A inhibitor may be at least two times or three times greater than an H-score for an autologous non-tumor tissue sample.

In addition to the H-score method, other scoring methods, such as the Allred method or an IRS can also be used. The Allred score is a scoring system which considers the percentage of cells that test positive for an analyte, along with the staining intensity of the analyte. These values are then combined to score the sample on a scale from 0 to 8. The IRS is calculated based on a scale reflecting the percentage of positive cells (on a scale of 1-4, where 0=0%, 1=<10%, 2=10%-50%, 3=>50%-80%, and 4=>80%) multiplied by the intensity of staining (on a scale of 1-3, where 1=weak, 2=moderate, and 3=strong). Immunoreactive scores range from 0-12. An appropriate minimum or “cut-off” score can be identified for each scoring method. For example an Allred score of at least 3, at least 4, at least 5, at least 6, at least 7, or at least 8 may be used as a minimum identifying score. In another example, an IRS of at least 2, at least 3 or at least 4 may be used as a minimum identifying score.

In certain of the foregoing embodiments, the Aurora kinase A inhibitor treatment can comprise alisertib. Alisertib is an orally bioavailable and highly selective small molecule inhibitor of Aurora A kinase. Alisertib binds to and inhibits Aurora A kinase, which may result in disruption of the assembly of the mitotic spindle apparatus, disruption of chromosome segregation, and inhibition of cell proliferation. Aurora A kinase localizes to the spindle poles and to spindle microtubules during mitosis, and is thought to regulate spindle assembly. Without being bound to a particular theory, it is postulated that alisertib may also bind or otherwise directly interact with CBX2.

The present disclosure encompasses tools that may be used to facilitate identification or classification of subjects as described above. For example, a kit may comprise an article that provides a scale of immunohistochemistry scores for reference by a user. The scale can additionally indicate a minimum score that identifies a sample having that score as coming from a tumor that is likely responsive to a treatment with an Aurora kinase inhibitor. In some embodiments the article is a printed material such as a card, a pamplet, an insert, a sticker, or a magnet. In certain embodiments, the scale presented by the article can comprise one or more of H-scores, Allred scores, and immunoreactive scores. In particular embodiments, the scale can comprise H-scores, and indicate a minimum identifying score of about 90 to about 300.

### Definitions

Biomarker may refer to any measurable indicator of the state of a cell, tissue, organ, or subject. In some embodiments, the biomarker is a molecule associated with a cell, for example a protein, peptide, or mRNA transcript. In some embodiments, the protein may be full-length or truncated protein, or a peptide fragment of a full-length protein. In some embodiments, a biomarker may be a nucleic acid, such as an mRNA transcript. In many embodiments, the biomarker may be an mRNA or protein associated with a given gene, for example CBX2, SOX4 MYLK, NOG, and TNFSF10. In most embodiments, the biomarker is a mammalian biomarker, for example human biomarker.

Subject includes any mammal, including mice, rats, guinea pigs, rabbits, dogs, cats, cows, horses, monkeys, and humans. A patient is a subject undergoing treatment or observation for a condition or disease, such as cancer. Cancer includes conditions or diseases of mammals characterized by uncontrolled cellular growth, hyperproliferative growth, hyperplasic growth, neoplastic growth, cancerous growth or oncogenic processes. Cancer may also refer to tumors, metastatic tissues or malignantly transformed cells, tissues, or organs, irrespective of histopathologic type or stage of invasiveness.

Chemotherapeutic agents include synthetic, natural, semisynthetic compounds and molecules useful in treating, killing, suppressing, or controlling cells that are displaying uncontrolled growth. Chemotherapeutic agents may reduce the proliferation of such cells and/or induce their death (programmed or non-programmed cell death). Chemotherapy refers to administration of such agents to a patient in need thereof. Chemoresistance may refer to the ability of a cell to avoid death or a decrease in proliferation in the presence of a chemotherapeutic agent.

Biological sample may refer to any sample removed, extracted, or derived from a patient or subject. In many embodiments a biological sample includes tissues, cells, protein, nucleic acids, etc. A biological sample may be processed for viewing or analysis, such as protein or nucleic acid quantitation. A control sample or reference sample may be a sample obtained from a similar subject, tissue, or cell that does not have, or is not expected to have, a disease or condition that is being assayed. In some embodiments, a reference sample may include samples from two or more subjects.

Therapeutically effective, as used herein refers to a therapeutic treatment, for example administration of a chemotherapeutic agent, that is adequate to accomplish a desired, expected, or intended result. When used in the context of treating a patient or subject, it may refer to an amount of agent which, when administered to a subject or patient for treating or preventing a disease, is an amount sufficient to effect such treatment or prevention of the disease or condition, and/or ameliorates at least one consequence of the disease or condition.

Inhibition or repression, as used herein means to lessen by some amount. For example, inhibition of gene expression by a compound or agent may cause the amount of protein or transcript in a given cell to be reduced relative to a cell that has not been contacted with the compound or agent.

Antibody may refer to any natural, synthetic, human, non-human, or humanized protein that may bind to an epitope on a target antigen. Antibodies may be mono or bi-specific. Antibodies may be multi or single chain proteins. In some embodiments the antibodies may include one or more Fc domains, Fv domains and/or CDRs. Antibodies may refer to peptides or peptide mimetics that bind to a target protein.

RNA interference (RNAi) includes any method of targeting expression of one or more genes by degrading mRNA transcribed from the gene. RNAi include siRNA, miRNA, shRNA, ribozyme, aptamers and the like, which typically contain some amount of sequence complementary to the gene transcript. RNAi techniques and methods are well known to those of skill in the art.

Peptide, polypeptide, and protein fragment may refer to natural or synthesized compounds and molecules containing natural or synthetic amino acids, amino acid equivalents and/or other non-amino groups. Peptides may be modified by replacement of one or more amino acids with related compounds, and/or modified by removing or adding elements, compounds, or molecules to one or more side chains or functional groups. The peptides can be linear or cyclic. Peptidemimetic refers to a compound or molecule that mimics a peptide in the peptide's ability to assume a three-dimensional structure on its own, or as a result of binding or contacting an epitope.

Metastasize may refer to the ability of a cancer cell to travel from the primary tumor, proliferate, and establish a second tumor. In some embodiments, a metastatic cell may be able to proliferate in suspension without attaching or adhering to a substrate, for example connective or other solid non-cancerous tissue.

### EXAMPLES

### Example 1—CBX2 is Upregulated in High Grade Serous Ovarian Cancer and is Associated with Poor Survival

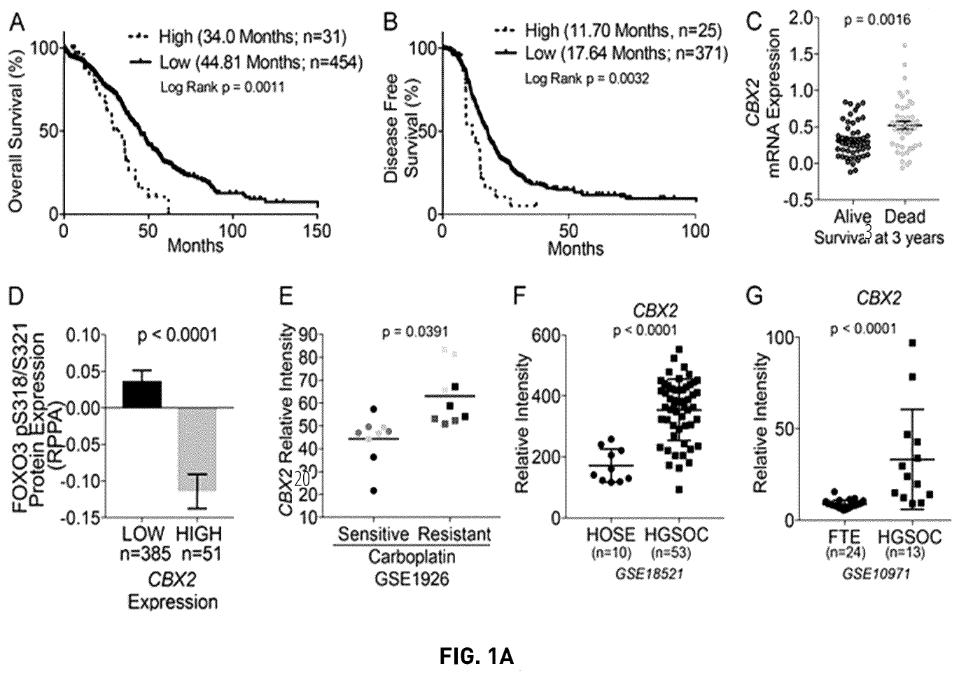

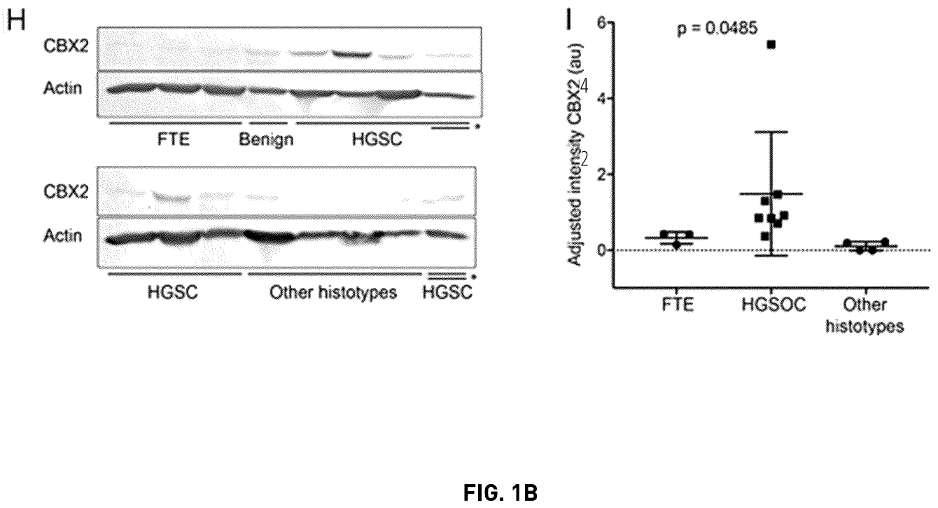

We examined CBX2 expression in HGSOC in several publicly available datasets (Gene Expression Omnibus; GEO Dataset and The Cancer Genome Atlas; TCGA). High expression of CBX2 in TCGA HGSOC samples conveyed both a significantly worse disease-free survival (DFS; 11.7 vs. 17.6 months, Log-rank test p-value 0.00316) and overall survival (OS; 34 vs. 44.8 months, Log-rank test p-value 0.00116) (FIG. 1A Panels A and B). In an independent HGSOC data set, high CBX2 expression was associated with poorer survival at 3 years (FIG. 1A Panel C). Further correlation of CBX2 expression with protein expression via reverse-phase protein array (RPPA) found several proteins significantly enriched or depleted in CBX2 high expressing tumors (Table 2). Notably, phosphorylated serine 318 and 321 FOXO3, a known tumor suppressor, was depleted in tumors with high CBX2 expression (FIG. 1A Panel D). Additionally, using the GEO Dataset (GSE1926), a comparison of platinum sensitive HGSOC tumors to platinum resistant HGSOC tumors, demonstrated an increase in CBX2 in resistant tumors, further supporting the association between CBX2 and more aggressive HGSOC (FIG. 1A Panel E).

TABLE 2

| | | | | Tendency in CBX2 |
|---|---|---|---|---|
| Protein | Log Ratio | p-Value | q-Value | High Tumors |
| TGM2 | −0.27 | 2.54E−05 | 5.25E−03 | Under-expressed |
| PEA15 | −0.31 | 8.81E−05 | 9.12E−03 | Under-expressed |
| FOXO3_PS318_S321 | −0.16 | 5.68E−04 | 0.0318 | Under-expressed |
| SCD | 0.27 | 7.26E−04 | 0.0318 | Over-expressed |
| INPP4B | −0.25 | 7.68E−04 | 0.0318 | Under-expressed |
| EIF4G1 | 0.37 | 9.56E−04 | 0.033 | Over-expressed |
| EIF4EBP1_PS65 | 0.18 | 1.39E−03 | 0.0412 | Over-expressed |

The panels of FIG. 1A and FIG. 1B are as follows. Panel A: Overall survival analysis comparing expression of CBX2 (High=mRNA expression>1.5 standard deviation; Low=SD<1.5 (=)). High (n=31) vs. Low (n=454) CBX2 expression (mean survival 34.0 months vs. 44.81 months, Log Rank p=0.0011). Panel B: Same as (Panel A), disease-free survival analysis, again comparing upregulation of CBX2, defined as above (High n=25, Low n=371). Increased expression of CBX2 was associated with statistically significant decreased disease-free survival (11.70 months vs. 17.64 months, Log Rank p=0.0032). Number of patients differ from (Panel A) due to available data within TCGA. Panel C: Examination of CBX2 mRNA expression in HGSOC patients alive (n=51) and dead (n=42) at 3 years after diagnosis. Data obtained from HGSOC Tothill Cohort. Statistical test=two-sided t-test, F test p=0.0057. Panel D: Correlation of CBX2 expression with FOXO3_PS318/321 in TCGA (“Provisional data” with RPPA data, n=435) tumors (High=upper quartile and Low=bottom three quartiles). Statistical test=two-sided t-test, F test p<0.0001. Panel E: Bioinformatic analysis evaluating relative intensity of CBX2 in high grade serous ovarian carcinoma (HGSOC) cases described as platinum sensitive (n=3) or resistant (n=3). Each tumor (color-coded) was examined in triplicate. Statistical test examined average CBX2 intensity for each tumor (GSE1926; one-sided t-test p=0.0391, F test p=0.29). Panel F: Relative intensity of CBX2 in benign human ovarian surface epithelium (HOSE, n=10) compared to HGSOC, n=53 (GSE18521; t-test p<0.0001, F test p<0.0001). Panel G: Relative intensity of CBX2 in fallopian tube epithelium (FTE, n=24) compared to HGSOC, n=13 (GSE10971; two-sided t-test p<0.0001, F test p<0.0001). Panel H: Protein lysates generated from the primary tissue of FTE, HGSOC, and mixed histotypes. Protein utilized for immunoblot against CBX2. (beta-actin=loading control). Panel I: Densitometric analysis of immunoblots. Intensities were normalized between immunoblot by indicated (*) sample (FTE vs. HGSOC, one-sided Rank Sum p=0.0333)

Ovarian surface and FTE are proposed to be the precursor cells for HGSOC; more recent data strongly support FTE as the predominant site of origin. Comparing CBX2 expression in ovarian surface epithelium or FTE to CBX2 expression in HGSOC, we observed that CBX2 was significantly higher in HGSOC (FIG. 1A Panels F and G) (GSE18521 and GSE10971). To confirm the extent of these findings we examined protein derived from primary tissues of four FTE and benign tissues and seven HGSOC tumors collected through the University of Colorado Gynecologic Tumor and Fluid Bank (GTFB) (FIG. 1B Panel H; Table 1). Utilizing densitometry, CBX2 expression was observed to be significantly higher in HGSOC primary tumor compared to FTE or benign tissues (FIG. 1B Panel I, Rank-sum test p value 0.0333). In other ovarian cancer histosubtypes, we noted a lack of CBX2 expression suggesting the oncogenic effects of CBX2 are HGSOC specific. Taken together, these data demonstrate that CBX2 upregulation in HGSOC is associated with poorer prognosis, repression of the FOXO3 tumor suppressor, and is possibly linked to chemoresistance.

### Example 2—CBX2 is Upregulated in Tumor Cells in Suspension

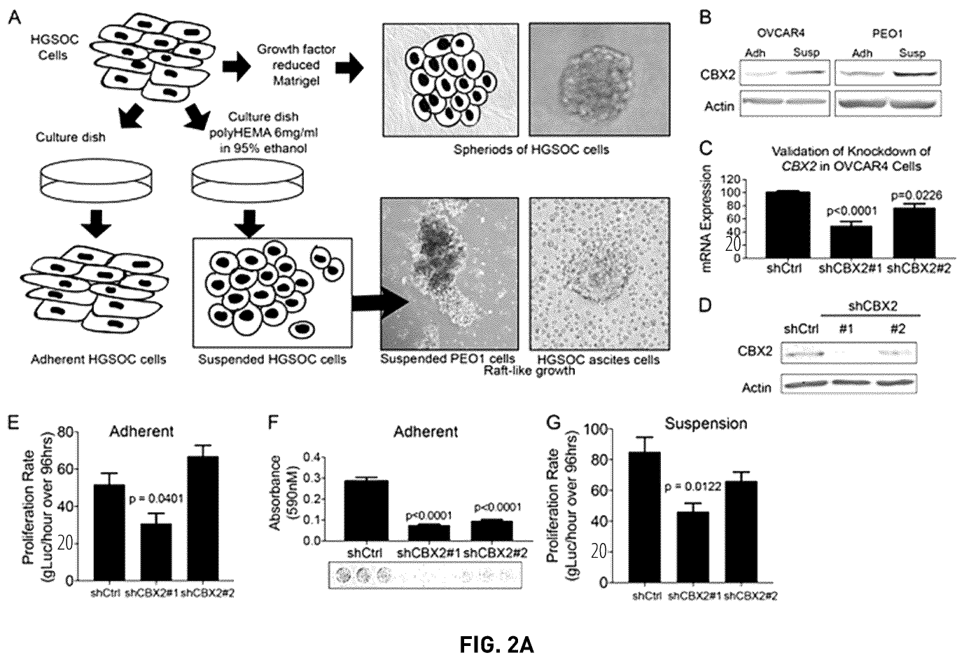

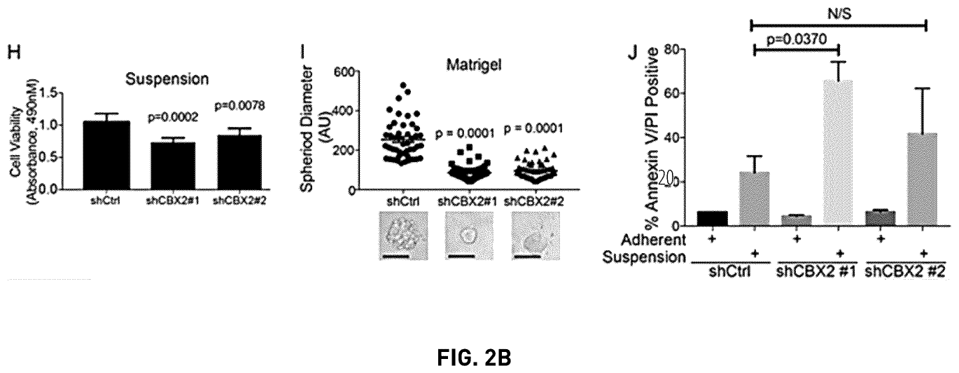

HGSOC is unique compared to other solid types in its tendency to directly seed and disseminate throughout the peritoneal cavity, which requires an escape from anoikis, an anchorage-independent cell death. To determine whether CBX2 plays a role in HGSOC tumor cell's ability to survive without anchorage, or in a suspended setting, we examined the role of CBX2 on HGSOC growth in suspension. A forced suspension setting was achieved by plating cells on polyHEMA-coated tissue culture dishes (FIG. 2A Panel A).

The panels of FIG. 2A and FIG. 2B are as follows. Panel A: Model describing basic protocol for establishing adherent, suspension, and spheroid growth environments. For adherent and suspension, two verified high grade serous ovarian carcinoma cell lines were initially grown on tissue culture plastic, then distributed to normal tissue culture dishes (adherent) and polyHEMA coated culture dish (suspension) growth environments. Distributed at 1:3 or 1:5 ratio to account for forced suspension induced cell death. Photographs show PEO1 cells after 7 days in suspension (left) and HGSOC cells directly derived from patient ascites. For spheroid formation, cells were grown in 3D in Matrigel for 12 days. A representative image of a resulting spheroid is shown at upper right. Panel B: Immunoblots against CBX2 protein from OVCAR4, and PEO1 cells grown in adherent and suspension settings (described in (Panel A)) over 7 days. Panel C: RT-qPCR for CBX2 in OVCAR4 cells transduced with small hairpin RNA (shRNA) specific for CBX2. Representative mRNA expression of CBX2 in shControl, shCBX2 #1, and shCBX2 #2. Statistical test=ANOVA. Panel D: Immunoblots against CBX2 protein derived from OVCAR4 shControl, shCBX2 #1, and shCBX2 #2 transduced cells (beta-actin=loading control). Panel E: Proliferation assay of OVCAR4 cells with CBX2 knockdown and shControl, grown in adherent setting (tissue culture plastic) over 96 h, evaluated using gLuc activity. Statistical test=ANOVA. Panel F: Same as (E), crystal violet staining and subsequent measurement of absorbance at 590 nm. Images of representative of stained cells from shControl, shCBX2 #1, and shCBX2 #2. Statistical test=ANOVA. Panel G: Proliferation assay of OVCAR4 cells with CBX2 knockdown and shControl, grown in suspension setting (poly-HEMA coated tissue culture plastic) over 96 h, evaluated using gLuc activity. Statistical test=ANOVA. Panel H: Same as (G), but cell viability was assessed via MTT after 96 h. Statistical test=ANOVA. Panel I: OVCAR4 cell lines (shControl, shCBX2 #1 and #2) grown in 3D using Matrigel over 12 days, leading to spheroid growth. Representative images of transduced OVCAR4 cells below. Scale bar=100 μm. Spheroids measured across horizontal diameter. Diameter mean calculated from measurements of 50 spheroids per cell type. Statistical test=ANOVA. Panel J: OVCAR4 cells with CBX2 knockdown and shControl grown in adherent or suspension. Cells were subjected to AnnexinV/PI apoptosis assay. Statistical test=two-sided t-test, F test p=0.9291. Error bars=S.E.M.

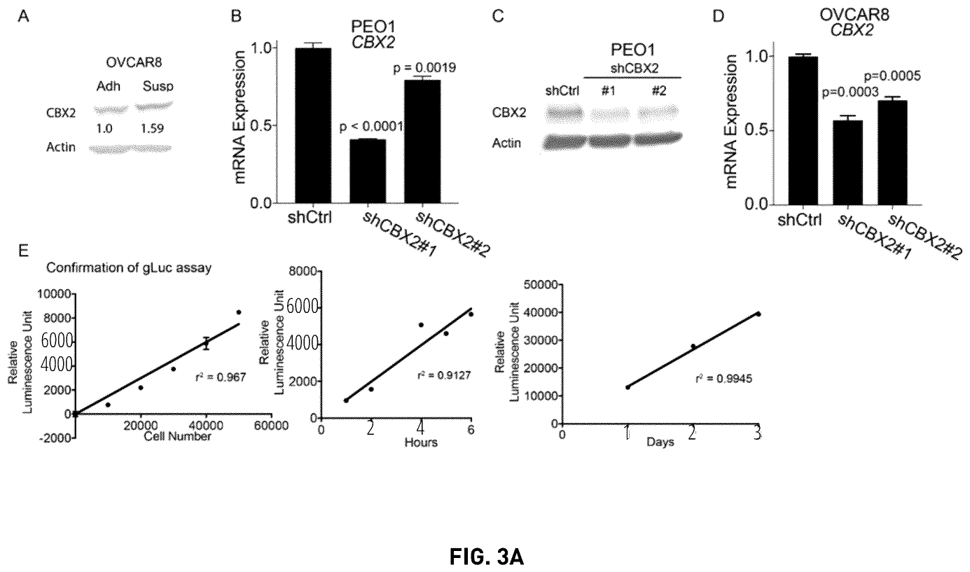

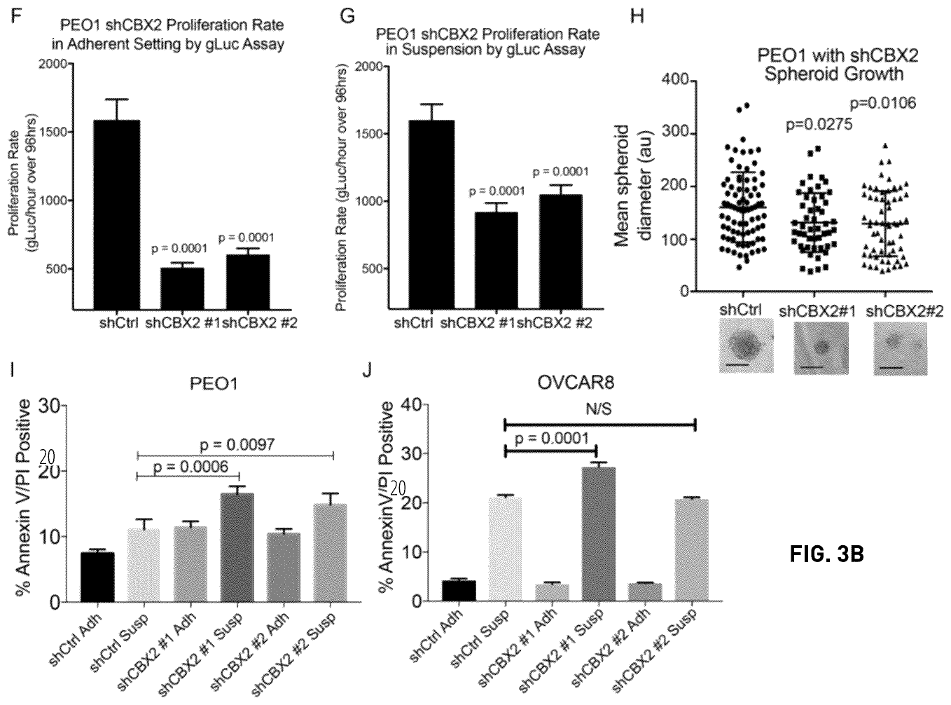

The high grade serous ovarian cancer cell lines OVCAR4, PEO1, and OVCAR8 were grown in adherent and suspended settings (FIG. 2A Panel A). During the course of optimizing our model, we noted that OVCAR4 and PEO1 cells grown in suspension demonstrated a morphology and organization similar to cells derived from primary ascites fluid (data not shown and FIG. 2A Panel A). After culturing HGSOC cell lines for 7 days in a forced suspension, condition protein was extracted and subsequently used for immunoblots against CBX2. For all cell lines examined, CBX2 expression was increased in cells grown in suspension conditions (FIG. 2A Panel B; and FIG. 3A Panel A). This observation serves as the foundation for our work, as the phenotype we describe is reinforced by both cell survival in suspension, as well as intact expression of CBX2.

The panels of FIG. 3A and FIG. 3B are as follows. Panel A: OVCAR8 grown in adherent and suspension settings (described in FIG. 2A) over 7 days. Protein utilized for immunoblot against CBX2. Panel B: RT-qPCR for CBX2 in PEO1 cells transduced with small hairpin RNA control (shCtrl) or specific for CBX (shCBX2 #1 and #2). Internal control=18s. Statistical test=ANOVA. Panel C: Protein derived from PEO1 shControl (shCtrl), shCBX2 #1, and shCBX2 #2 transduced cells utilized for immunoblot against CBX2 (actin=loading control). Panel D: Same as B, but examined CBX2 mRNA expression in OVCAR8 cells. Internal control=18s. Statistical test=ANOVA. Panel E: Experimental confirmation of Gaussia luciferase (gLuc) assay. OVSAHO cells were transduced with gLuc virus and selected with puromycin. Known number of cells grown over 24 hours, media collected, gLuc assay performed (left). Equivalent number of cells seeded across 24 well plate, media collected and assayed over hours (middle) and days (right). Relative luminescence units by gLuc increases with number of cells. Linear regression r² indicated. Panel F: Proliferation assay of PEO1 cells with CBX2 knockdown and scramble control, grown in adherent setting (tissue culture plastic), measured by gLuc activity every 24 hr for 96 hr. Calculated as proliferation rate: gLuc relative intensity per hour. Statistical test=ANOVA. Panel G: Same as E, PEO1 cells grow in suspension setting with utilization of polyHEMA coated plates (FIG. 2A Panel A). Proliferation rate calculated. Statistical test=ANOVA. Panel H: PEO1 cell lines (shControl [shCtrl], shCBX2 #1 and #2) grown in 3D using Matrigel over 12 days, forcing spheroid growth. Spheroids measured across horizontal diameter. Mean calculated from all measurements. Representative images of spheroids below X axis. Scale Bar=100 μm. Statistical test=ANOVA. Panel I: ShCtrl, shCBX2 #1 and #2 PEO1 cells grown in adherent or suspension were utilized for an AnnexinV/PI assay. Percentage positive AnnexinV/PI graphed. Statistical test=ANOVA. Panel J: Same as I, but examined OVCAR8 cells. Error bars=S.E.M.

### Example 3—CBX2 knockdown Inhibits Proliferation

To further elucidate the role of CBX2 in HGSOC, we evaluated the impact of CBX2 modulation in PEO1, OVCAR4, and OVCAR8 HGSOC cell lines. One of the two independent small hairpin RNAs (shRNA) specific for CBX2 or a control (shControl) were transduced into OVCAR4, PEO1, and OVCAR8 cells. CBX2 knockdown was confirmed via quantitative PCR (qPCR) and immunoblots, with approximately 60% knockdown in the presence of shCBX2 #1 and 30% knockdown in the presence of shCBX2 #2 (FIG. 2A Panels C, D; and FIG. 3A Panels B-D). PEO1 and OVCAR4 CBX2 knockdown cells were subjected to proliferation assays in 2D tissue culture dishes and in suspension as demonstrated in FIG. 2A Panel A. To assess changes in proliferation, cells were transduced with a retrovirus specific for Gaussia luciferase (gLuc). Changes in gLuc activity were shown to be directly correlated with cell number (FIG. 3A Panel E). OVCAR4 and PEO1 CBX2 knockdown cells were plated in adherent (2D) conditions and for 96 h gLuc activity was measured every 24 h. As a confirmatory assay, colony formation was examined in parallel on cells grown in 2D. CBX2 knockdown cells had a significantly reduced rate of gLuc activity and reduced colony formation (FIG. 2A Panels E and F and FIG. 3B Panel F). OVCAR4 and PEO1 CBX2 knockdown cells were plated in forced suspension conditions and gLuc activity was monitored every 24 h for 96 h and cell viability was determined for cells grown in forced suspension. Similar to adherent conditions, CBX2 knockdown had a significantly reduced rate of gLuc activity and viability (FIG. 2A Panel G, FIG. 2B Panel H and FIG. 3B Panel G). HGSOC grown on extracellular matrix more closely recapitulates the tumor microenvironment, therefore OVCAR4 and PEO1 shControl and shCBX2 (#1 and #2) cells were grown in matrigel for 12 days. Spheroid diameter was measured for at least 50 spheroids in each condition and used as a surrogate for cell number. CBX2 knockdown significantly reduced spheroid size compared to shControl control cells (FIG. 2B Panel I and FIG. 3B Panel H). In all culture conditions examined, we observed that CBX2 knockdown significantly decreased HGSOC cell viability. Upon closer examination of forced suspension conditions, we observed in OVCAR4, PEO1, and OVCAR8 cells that CBX2 knockdown potentiated anoikis, anchorage-independent cell death (FIG. 2B Panel J and FIG. 3B Panels I-J). These data indicate that CBX2 is important in promoting HGSOC cell proliferation and protecting against anoikis.

### Example 4—Tissue Microarray Supports a Role for CBX2 in Tumor Progression

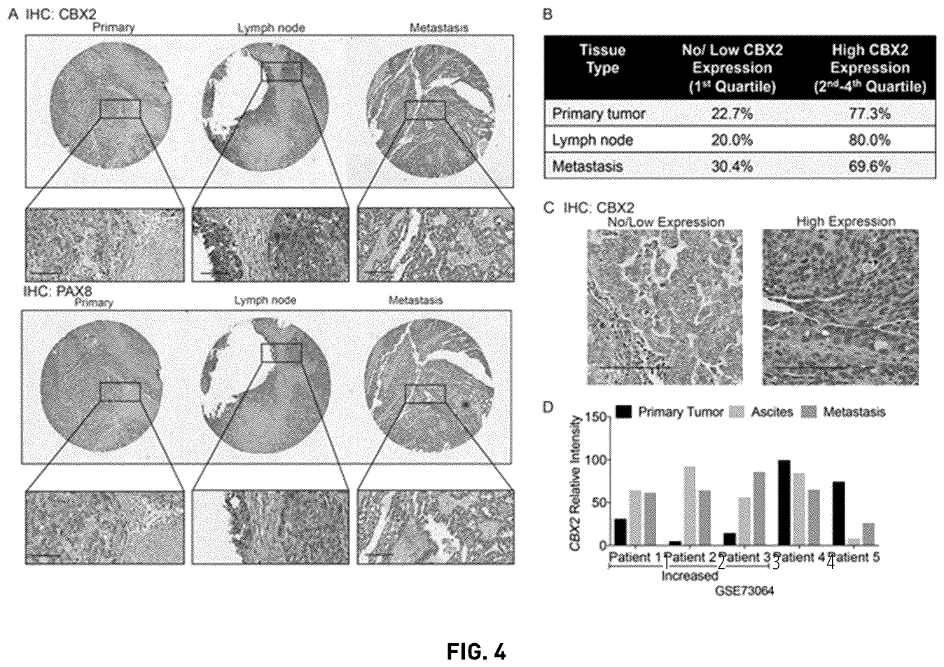

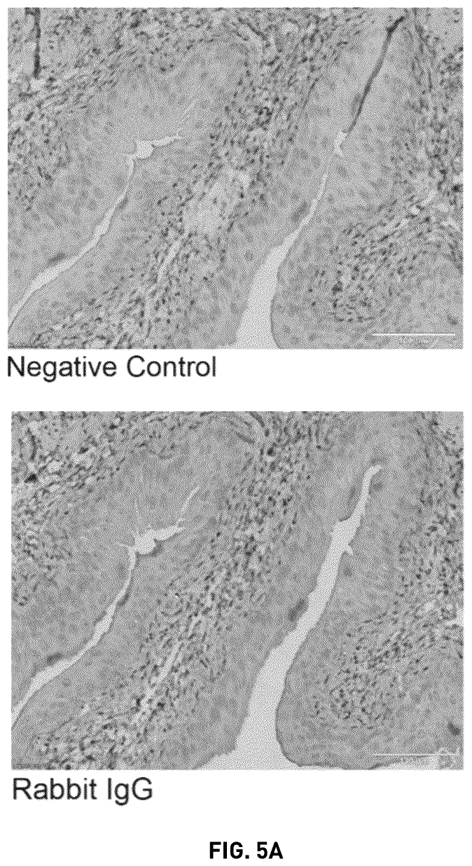

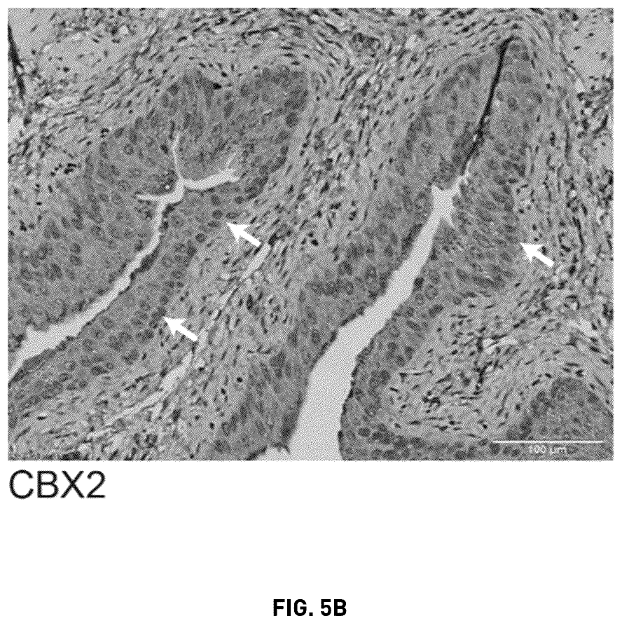

In order to correlate the CBX2 in vitro findings to clinically relevant specimens, we utilized a tissue microarray (TMA) of HGSOC that recapitulated tumor progression. The TMA contained 24 primary tumors, with matched lymph node or distant metastases (Table 3). IHC was optimized and performed using a previously published CBX2 antibody, predicted to preferentially stain the nucleus (FIG. 4 Panel A, and FIG. 5A and FIG. 5B).

TABLE 3
Clinical characteristics of matched tissue microarray

| | | | | % Positive | % Positive | % Positive |
|---|---|---|---|---|---|---|
| Age at | Stage at | Pathologic | | Stain | Stain | Stain |
| diagnosis | diagnosis | diagnosis | PDS vs NACT | Primary | Lymph node | Metastasis |
| No clinical | | | | 36.51% | 22.09% | 27.44% |
| data available | | | | | | |
| No clinical | | | | 20.68% | 26.93% | 33.06% |
| data available | | | | | | |
| No clinical | | | | No | No | 45.85% |
| data available | | | | primary | primary | |
| 36 | IV | [Serous carcinoma, | PDS (optimal) | 34.30% | 53.14% | 10.75% |
| | | FIGO Grade III per | | | | |
| | | clinical | | | | |
| | | documentation] | | | | |
| 59 | IIIC | Papillary serous | PDS (optimal) | 50.23% | 29.23% | 24.21% |
| | | carcinoma | | | | |
| 84 | IIIC | Papillary serous | PDS (optimal) | 32.84% | 23.91% | 18.66% |
| | | carcinoma | | | | |
| 46 | IIIC | Papillary serous | PDS (optimal) | 14.40% | 26.76% | 15.55% |
| | | carcinoma | | | | |
| 73 | IIIC | Serous carcinoma, | PDS (optimal) | 6.96% | n/a | 18.72% |
| | | FIGO Grade III | | | | |
| No clinical | | | | 32.60% | 50.16% | n/a |
| data available | | | | | | |
| 67 | IIIC | Poorly differentiated | PDS (optimal) | 26.75% | n/a | 13.88% |
| | | high-grade (FIGO III) | | | | |
| | | multifocal serous | | | | |
| | | cystadenocarcinoma | | | | |
| 39 | IV | Moderate to poorly | PDS (optimal) | 30.24% | 23.62% | 30.78% |
| | | differentiated | | | | |
| | | serous papillary | | | | |
| | | cystoadenocarcinoma | | | | |
| | | (FIGO III) | | | | |
| 44 | IIIC | Serous carcinoma, | PDS (optimal) | 2.77% | 3.43% | 11.49% |
| | | FIGO grade III | | | | |
| 50 | IIIC | Papillary serous | PDS (optimal) | 14.96% | n/a | 12.52% |
| | | carcinoma, high grade | | | | |
| 63 | IIIC | Poorly differentiated | PDS (optimal) | n/a | 16.82% | n/a |
| | | serous carcinoma | | | | |
| 54 | IIIC | Serous carcinoma | PDS (optimal) | 33.47% | 9.53% | 31.03% |
| | | (FIGO Grade III) | | | | |
| 74 | IIIC | Poorly differentiated | PDS (optimal) | n/a | 13.43% | 27.78% |
| | | serous carcinoma, | | | | |
| | | Grade III | | | | |
| 66 | IVA | Serous carcinoma, | PDS (optimal) | 19.07% | 34.20% | 2.76% |
| | | Grade III | | | | |
| No clinical | | | | 19.27% | 27.81% | 17.52% |
| data available | | | | | | |
| 54 | IIIC | High grade serous | PDS (optimal) | 31.67% | 28.51% | 25.01% |
| | | carcinoma | | | | |
| 50 | IIIC | Poorly differentiated | PDS (optimal) | 27.17% | 45.78% | 25.94% |
| | | serous carcinoma | | | | |
| 60 | IVB | High grade serous | PDS | 23.51% | 18.58% | 24.09% |
| | | carcinoma | (suboptimal) | | | |
| 51 | IIIC | High grade serous | PDS (optimal) | 67.66% | 38.10% | 33.35% |
| | | papillary carcinoma | | | | |
| 66 | IVB | High grade serous | PDS (optimal) | 42.95% | 50.99% | 32.12% |
| | | carcinoma | | | | |
| 70 | IVA | Poorly differentiated | PDS | 15.81% | n/a | 23.21% |
| | | serous carcinoma | (suboptimal) | | | |
| 63 | IIIC | High grade serous | PDS | 36.87% | n/a | 25.52% |
| | | carcinoma | (suboptimal) | | | |

In parallel the TMA was stained with PAX8, a marker for Müllerian origin (FIG. 4). CBX2 and PAX8 stained TMAs were scanned using the Aperio system and annotated based on the PAX8 staining profile. Objective software-based approaches were utilized to score and analyze the scanned and annotated CBX2 TMAs. The level of expression was compared between primary tumors, metastases, and lymph nodes and quartiles were calculated. Across all three tissue types, 20-30% of the specimens were considered “no or low expression” (first quartile) compared to 69-80% of the tissues that were moderate to high expression (second through fourth quartile) (FIG. 4 Panels B and C). Results from Aperio analysis were confirmed via manual scoring by two independent board-certified pathologists. Similarly, when the intensity of staining was evaluated between matched samples there was no significant change between matched samples. Given that the tissues were derived from HGSOC patients with advanced disease, as demonstrated by both metastatic and lymph node involvement, this would suggest that CBX2 expression potentially correlates with a more aggressive disease. We next examined CBX2 expression in matched primary tumor, ascites-associated tumor cells, and metastatic tumors from five HGSOC patients (GSE73064). In two of the five patients, CBX2 expression was high in the primary tumor, however three of the five patients had an increase of CBX2 in metastatic and ascites samples compared to primary tumor (FIG. 4 Panel D). Consistently, these findings align with our bioinformatics analysis indicating that increased CBX2 expression is associated with decreased survival and more advanced disease.

FIG. 4 panels are as follows. Panel A: Immunohistochemistry (IHC) against CBX2 and PAX8 utilizing a HGSOC tissue microarray (TMA) of 24 matched patient samples. Representative images of matched patient samples shown. Initial images shown at 3×, inset of images at 18×. Scale bar=100 μm. Panel B: TMA analyzed using PAX8 staining a control for tumor area followed by analysis with Image Scope software to determine relative CBX2 tumor-associated intensity. Level of expression divided into quartiles. Breakdown of samples into 1st quartile (low or no expression) compared to 2nd-4th quartiles (moderate or high expression). Analysis by two board-certified pathologists (authors: M.D.P. and A.A.B.) confirmed Aperio findings. Values displayed in table, CBX2 expression seems to be consistent between tissue types. Panel C: Representative images of “High” and “No/Low” CBX2 expression. Scale bar=100 μm. Panel D: Bioinformatic analysis evaluating relative intensity of CBX2 in high grade serous ovarian carcinoma (HGSOC) cases with matched primary tumor (black bars), ascites-associated tumor cells (Ascites, light gray bars), and distant metastasis (dark gray bars) (n=5, GSE73064).

FIG. 5A and FIG. 5B show serial sections of a HGSOC tumors were utilized for IHC against a negative control, a matched isotype (Rb IgG), and CBX2 (brown). As expected a majority of the CBX2 localized to the nucleus (white arrows). Scale bars=100 μm.

### Example 5—CBX2 Expression is Associated with Chemoresistance

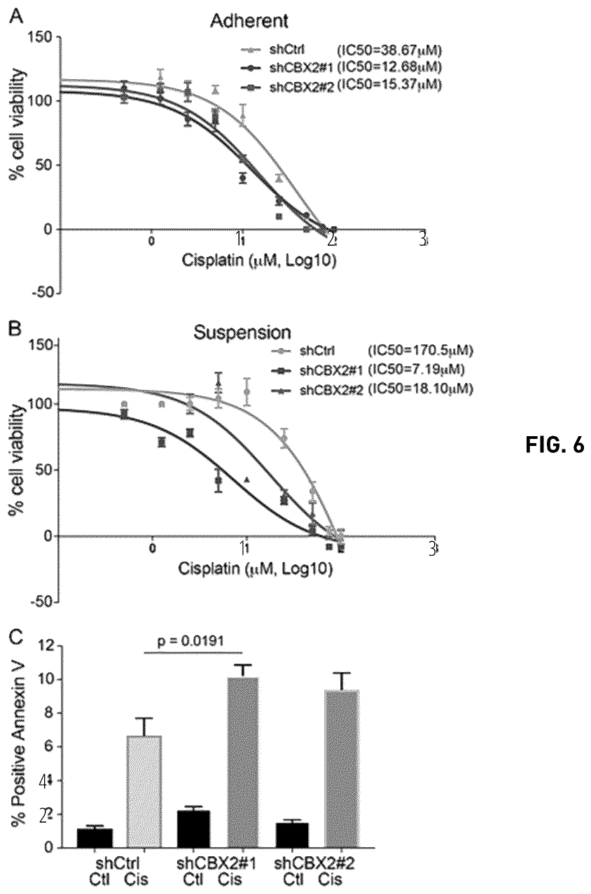

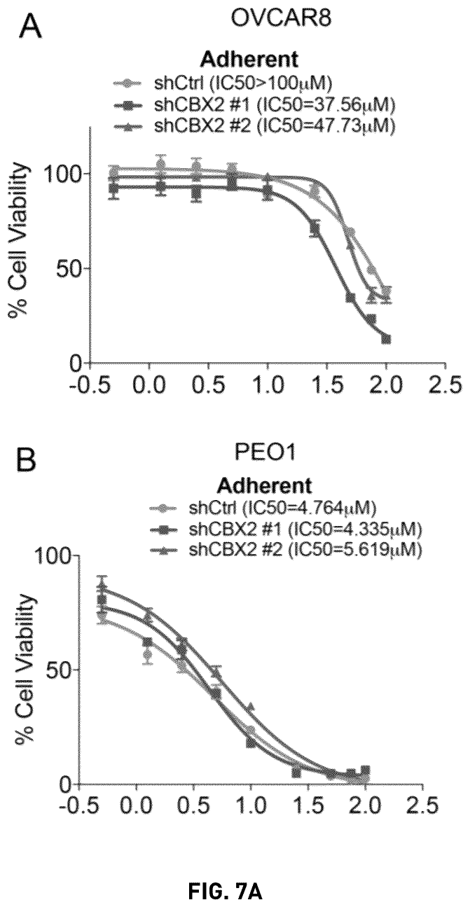

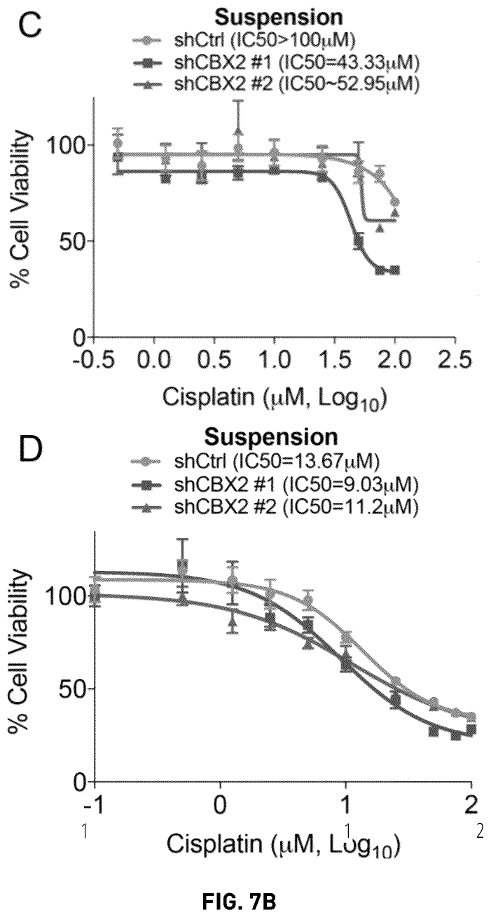

A majority of HGSOC patients treated with platinum-based (i.e., carboplatin) chemotherapy develop chemoresistance. A comparison of carboplatin sensitive HGSOC tumors to platinum resistant tumors demonstrates an increase in CBX2 in platinum resistant tumors (GSE1926) (FIG. 1A Panel C). Based on these data and our finding that CBX2 protects against anoikis, we hypothesized that CBX2 attenuates chemotherapy response. To test this hypothesis, CBX2 knockdown OVCAR4, PEO1, and OVCAR8 cells were grown in an adherent setting for 24 h and subsequently treated with increasing doses of cisplatin. Assessment of cell viability showed that in OVCAR4, PEO1, and OVCAR8, shCBX2 cell lines were significantly more chemosensitive than the shControl cells (FIG. 6 Panel A and FIG. 7A Panels A-B).

For example, CBX2 knockdown OVCAR4 cells had an IC50 of 12.68 μM in shCBX2 #1 and 15.37 μM in shCBX2 #2 compared to 38.67 μM for the control with intact CBX2 (FIG. 6 Panel A). Haley et al. reported the OVCAR4 cisplatin IC50 to be approximately 6 μM, however unlike this report we did not allow cells to recover 72 h following cisplatin treatment which likely accounts for this discrepancy. CBX2 knockdown OVCAR4, PEO1, and OVCAR8 cells were grown in suspension and dosed with cisplatin. OVCAR4 shCBX2 cell lines grown in suspension were found to be re-sensitized to platinum treatment with an IC50 of 7.19 μM (shCBX2 #1) and 18.10 μM (shCBX2 #2) compared to the control with intact CBX2 at an IC50 of 170.50 μM (FIG. 6 Panel B). Although not as robust as OVCAR4 cells, CBX2knockdown also sensitized OVCAR8 and PEO1 cells to cisplatin (FIG. 7B Panels C-D). Notably, in OVCAR4 cells we observed a 4.47-fold increase in the cisplatin IC50 in suspension cells compared to the adherent cells. In addition, we further confirmed that in OVCAR4, loss of CBX2 lead to increased cisplatin-induced apoptosis measured with Annexin V/PI (FIG. 6 Panel C). These findings strongly support the hypothesis that anoikis-resistance, demonstrated by survival in suspension, and intact CBX2 expression, both promote chemoresistance.

Panels of FIG. 6 are as follows. Panel A: OVCAR4 shControl, shCBX2 #1, and shCBX2 #2 in 96-well plates treated over 24 h with increasing dose of cisplatin (0.5-100 μM). Percent cell viability was measured using the MTT assay and the half maximal inhibitory concentration (IC50) calculated. Panel B: Similarly to (Panel A), OVCAR4 knockdown cell lines were grown in low adherent 96-well plates (forced suspension) and treated with increasing doses of cisplatin over 24 h and percent cell viability measured with MTT for calculation of IC50. Panel C: Annexin/V apoptosis assay of OVCAR4 cells grown in adherent setting with shControl, shCBX2 #1 or #2 treated with cisplatin (10 μM) compared to untreated control. Percent Annexin positive cells are shown. Statistical test=ANOVA. Error bars=S.E.M.

The panels in FIG. 7A and FIG. 7B are as follows. Panel A: shControl (shCtrl), shCBX2 #1 and #2 OVCAR8 cells grown in adherent were dosed with cisplatin for 24 hours. Treated cells were utilized for a MTT assay to assess cell viability. Panel B: Same as A, but adherent PEO1 cells were dosed with cisplatin for 48 hours. Panel C: Same as A, but OVCAR8 cells in suspension were dosed with cisplatin for 24 hours. Panel D: Same as A, but PEO1 cells in suspension were dosed with cisplatin for 48 hours. IC50 values were calculated with Prism and are indicated.

### Example 6—CBX2 Regulation of Autophagy, Apoptosis, and EMT-Related Genes

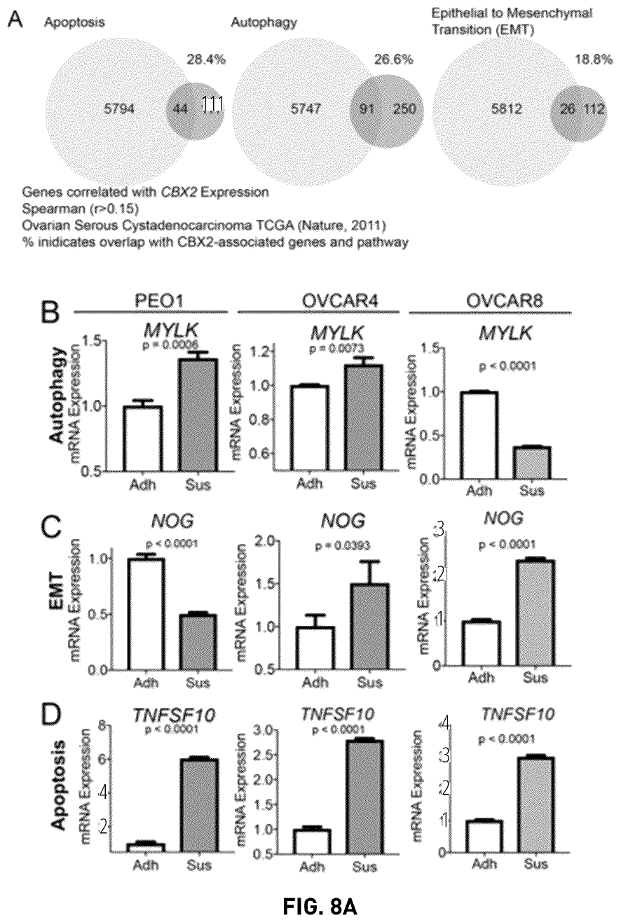

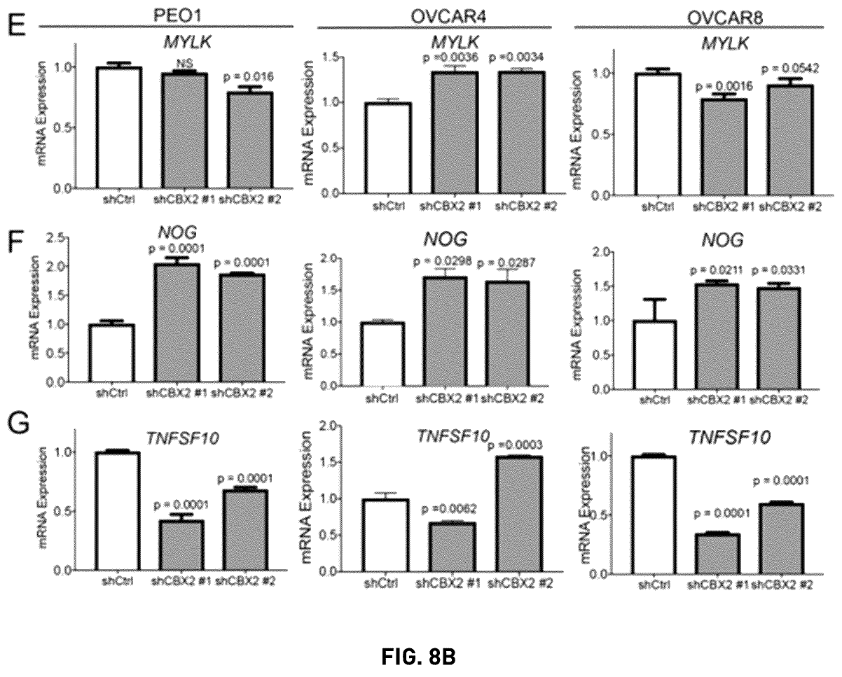

Chemoresistance and dissemination of ovarian cancer cells are associated with changes in apoptosis, epithelial to mesenchymal transition (EMT), and autophagy. The PcG is an epigenetic complex that is responsible for transcriptional reprogramming through histone modification thus CBX2 could regulate a variety of genes. Utilizing HGSOC TCGA data we generated a list of potential CBX2 target genes through examination of mRNA correlations (Spearman r>0.15, 5838 genes). Utilizing published gene sets for EMT, autophagy, stemness, and apoptosis we cross-referenced the CBX2-associated genes. CBX2-associated genes accounted for 18.8-28.4% of genes in the respective pathways (FIG. 8A Panel A). We selected a gene from each pathway and in OVCAR4, OVCAR8, and PEO1 observed that the genes were differentially regulated in adherent vs. suspension culture conditions. For instance, in two of the three cell lines an autophagy-related gene, MYLK, and EMT-related gene, NOG, were significantly differentially regulated in suspension (FIG. 8A Panels B, C). Furthermore, we examined an apoptosis-related gene, TNFSF10, and observed that it was upregulated in all three HGSOC cells lines when grown in suspension (FIG. 8A Panel D). Subsequently, CBX2 knockdown cells grown in suspension differentially regulate MYLK, NOG, and TNFSF10 (FIG. 8B Panels E-G). The differential expression observed correlated with the level of CBX2 knockdown (FIG. 2A Panel C and FIG. 3A Panels B-D). These findings suggest that in the context of anoikis CBX2 regulates several pathways, including autophagy, apoptosis, and EMT.

The panels of FIG. 8A and FIG. 8B are as follows. Utilizing the TCGA (HGSOC, Nature, 2011) dataset, CBX2 expression was correlated to mRNA expression of all genes and 5838 genes (CBX2-associated genes) were identified to have a Spearman correlation of greater than r=0.15 (Panel A). The 5838 genes were cross-referenced with published gene sets for “Apoptosis”, “Autophagy”, and “Epithelial to Mesenchymal Transition (EMT).” Percentage indicates overlap with gene lists. PEO1, OVCAR4, and OVCAR8 cells were grown in adherent (Adh) or suspension (Sus) for 7 days, RNA was extracted, and used for RT-qPCR against MYLK (Panel B), NOG (Panel C), and TNFSF10 (Panel D). Statistical test=two-sided t-test. shControl and shCBX2 #1 and 2 PEO1, OVCAR4, and OVCAR8 cells were grown in suspension, RNA was extracted, and used for RT-qPCR against MYLK (Panel E), NOG (Panel F), and TNFSF10 (Panel G). Statistical test=ANOVA. Error bars=S.E.M.

### Example 7—Forced Growth in Suspension and Increased CBX2 Leads to a Stem-Like Phenotype

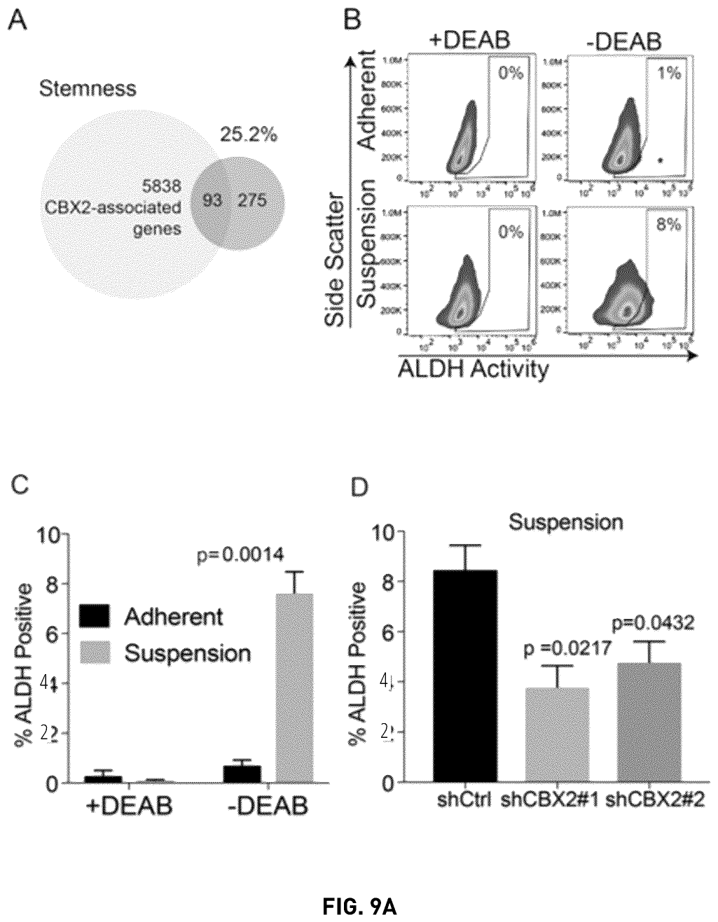

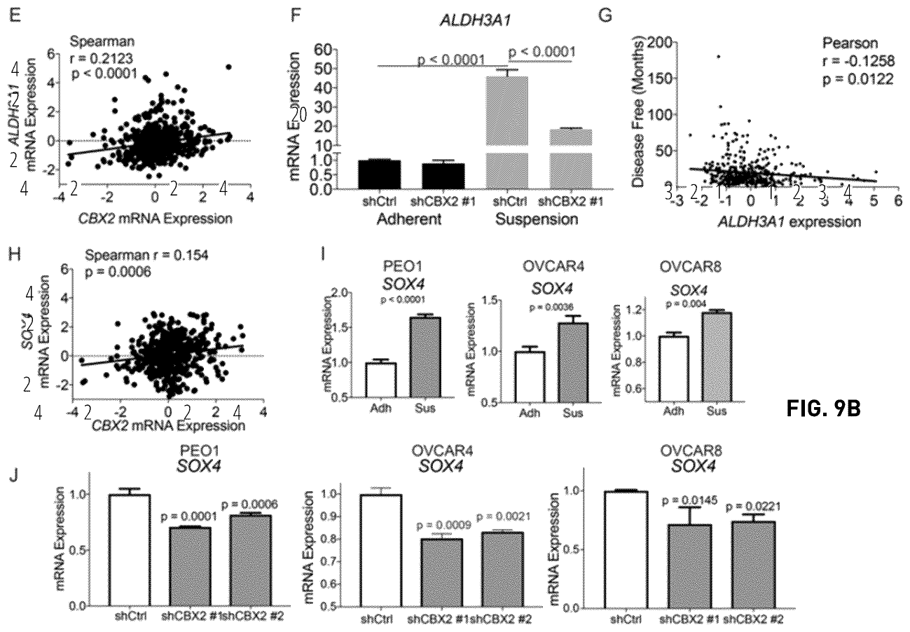

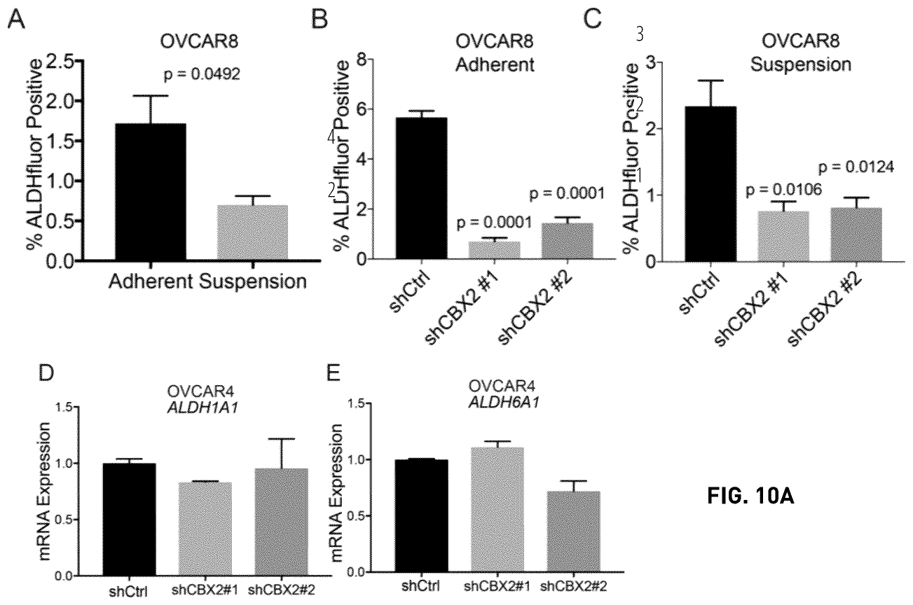

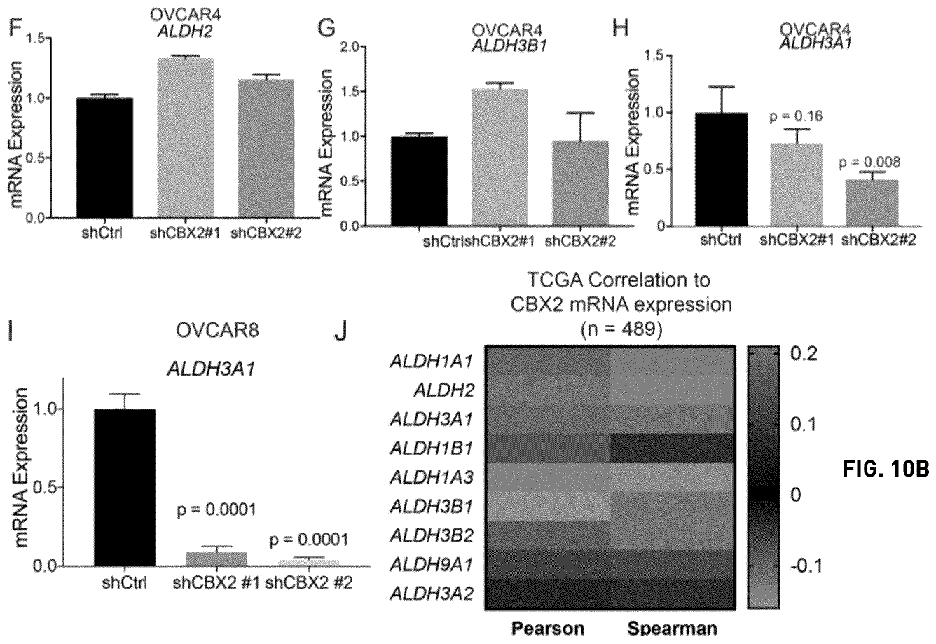

Several reports have demonstrated cells that survive in anchorage-independent conditions possess stem-like characteristics. We hypothesize that the polycomb repressor complex could be involved by inhibiting cellular differentiation and thus maintaining stemness. Applicants examined the CBX2-associated genes from TCGA against a published stemness gene set. We observed 25.2% of stemness-related genes overlapped with CBX2 genes. We next evaluated stemness by measuring aldehyde dehydrogenase (ALDH) activity both in the setting of suspension growth and CBX2 knockdown. We first determined whether placing OVCAR4 and OVCAR8 cells in suspension increased ALDH activity. We observed that in OVCAR4 cells ALDH activity was significantly increased in cells grown in suspension for 7 days (FIG. 9A Panels B and C). In contrast, we did not observe an increase in ALDH activity in OVCAR8 cells grown in suspension for 7 days (FIG. 10A Panel A).

The panels of FIG. 10A and FIG. 10B are as follows: Panel A: OVCAR8 cells were grown in adherent and suspension conditions for 7 days and utilized for ALDHfluor assay. Percentage of ALDH positive cell graphed. Statistical test=t-test. Panel B: shControl (shCtrl), shCBX2 #1 and #2 OVCAR8 cells were grown in adherent condition for 7 days and utilized for ALDHfluor assay. Percentage of ALDH positive cell graphed. Statistical test=ANOVA. Panel C: Same as B, but examined ALDH positive OVCAR8 cells grown in suspension for 7 days. Statistical test=ANOVA. Panel D: RT-qPCR for ALDH1A1 in OVCAR4 cells transduced with shControl (shCtrl) or shCBX2 #1 and #2. RNA was collected from adherent cells and used for RT-qPCR against CBX2. Panel E: Same as D, but RT-qPCR for ALDH6A1. Panel F: Same as D, but RT-qPCR for ALDH2. G: Same as D, but RT-qPCR for ALDH3B1. Panel H: Same as D, but RT-qPCR for ALDH3A1. Statistical test=ANOVA. I: Same as D, but examined OVCAR cells and RT-qPCR for ALDH3A1. Statistical test=ANOVA. Note: All RT-qPCR 18s was utilized as an internal control. Panel J: TCGA (Nature, 2011, n=489) analysis of HGSOC examining correlation between CBX2 expression and indicated ALDHs. Pearson and Spearman correlations (r values) shown in heatmap.

Next, we evaluated whether CBX2 knockdown impacted suspension-induced ALDH activity, we found that culturing OVCAR4 and OVCAR8 shCBX2 cells in suspension significantly inhibited ALDH activity compared to shControl cells (FIG. 9A Panel D and FIG. 10A Panel B-C). These results highlight that the loss of CBX2 regulates ALDH activity in OVCAR4 and OVCAR8 cell lines. The polycomb repressor complex 2 regulates ALDH1A1 expression, so we sought to determine whether this decrease in ALDH activity correlated with the expression of a specific ALDH gene. We examined the expression of ALHD1A1, ALDH2, ALDH3A1, ALDH3B1, and ALDH6A1 genes in CBX2 knockdown cells (FIG. 10A Panels D-E and FIG. 10B Panels F-H). ALDH1A1 was not significantly changed in shCBX2 cells. However, in OVCAR4 and OVCAR8 cells, shCBX2 knockdown cells ALDH3A1 expression was significantly decreased (FIG. 10B Panel H-I). Similar to ALDH1A1, ALDH3A1 has previously been associated with stemness. Consistently, the examination of HGSOC samples in the TCGA revealed CBX2 expression correlated with ALDH genes expression with the ALDH3A1 gene having the only significant positive correlation (FIG. 10B Panel J and Table 4). Further analysis of TCGA data comparing CBX2 and ALDH3A1 expression found a significant positive correlation; suggesting CBX2 promotes increased ALDH3A1 expression (FIG. 9B Panel E). In suspension, ALDH3A1 was significantly upregulated compared to adherent cells and knocking down CBX2 significantly abrogated ALDH3A1 expression (FIG. 9B Panel F and FIG. 10B Panels H-I). Furthermore, ALDH3A1 expression significantly correlated with disease-free survival (FIG. 8B Panel G, Pearson's r=−0.1258, p=0.0122). Referring to the TCGA data comparing CBX2 expression we also identified a significant correlation to a potent stem cell-associated transcription factor, SOX4 (FIG. 9B Panel H). In adherent vs. suspension settings SOX4 was significantly upregulated in OVCAR4, OVCAR8, and PEO1 cells grown in suspension (FIG. 9B Panel I). Consequentially, CBX2 knockdown promoted a significant decrease in SOX4 expression (FIG. 9B Panel J). Taken together, these data strongly suggest that HGSOC cells grown in suspension are more stem-like and CBX2 could be promoting stemness through ALDH3A1 and SOX4 regulation.

TABLE 4

| | Pearson | Spearman |
|---|---|---|
| | Correlation | Correlation |
| Gene | (r) | (r) |
| ALDH1A1 | −0.1 | −0.13 |
| ALDH2 | −0.12 | −0.14 |
| ALDH3A1 | 0.19 | 0.21 |
| ALDH3B1 | −0.08 | −0.12 |
| ALDH1A3 | −0.14 | −0.15 |
| ALDH3B1 | −0.16 | −0.12 |
| ALDH3B2 | −0.09 | −0.12 |
| ALDH9A1 | −0.06 | −0.07 |
| ALDH3A2 | −0.03 | −0.04 |

TCGA Data (n = 489, Nature, 2011)

The panels of FIG. 9A and FIG. 9B are as follows. Panel A: CBX2-associated genes were cross-referenced with a gene set for stemness. Percentage indicates overlap of stemness gene set with CBX2-associated genes. Panel B: OVCAR4 cells grown in adherent and suspended settings for 7 days. Aldefluor assay and flow cytometry were utilized to determine the percentage of cells that were positive for aldehyde dehydrogenase (ALDH), a marker of stemness. Diethylaminobenzaldehyde (DEAB), a potent ALDH inhibitor, prevented the increase in ALDH activity and served as negative control (left). Panel C: As above, OVCAR4 cells grown in adherent and suspended settings for 7 days. Bar graph compares the percentage of cells ALDH positive control (+DEAB) to cells without DEAB (experimental) in adherent and suspended settings. Statistical test=two-sided t-test. F Test p=0.1136. Panel D: OVCAR4 cells with shControl, shCBX2 #1 and #2 cultured in suspension over 7 days. Aldefluor assay and flow cytometry again utilized to determine the percentage of cells ALDH positive. Statistical test=two-sided t-test. F-test p=0.83 Panel E: Utilizing the TCGA (HGSOC, Nature, 2011, n=489) dataset, a scatter plot CBX2 expression was correlated to ALDH3A1 expression. Spearman correlation r=0.2123 and p-value<0.0001. Panel F: RT-qPCR of ALDH3A1 in OVCAR4 cells cultured in adherent and suspension conditions with CBX2 knockdown (shCBX2 #1). Statistical test =ANOVA. Panel G: Scatter plot of ALDH3A1 expression (x-axis, Z-score) compared to disease-free survival (y-axis, months). Pearson's correlation r=−0.1258 and p-value=0.0122. Panel H: Utilizing the TCGA (HGSOC, Nature, 2011, n=489) dataset, a scatter plot CBX2 expression was correlated to SOX4 expression. Spearman correlation r=0.154 and p-value=0.0006. Panel I: PEO1, OVCAR4, and OVCAR8 cells grown in adherent (Adh) or suspension (Sus) settings for 7 days. RNA was extracted, and used for RT-qPCR against SOX4. Statistical test=two-sided t-test. Experiment performed in technical triplicates and biological duplicate. Panel J: shControl and shCBX2 #1 and #2 PEO1, OVCAR4, and OVCAR8 were cultured in suspension conditions, RNA was extracted and used for RT-qPCR against SOX4. Experiment performed in technical triplicates and biological duplicate. Statistical test=ANOVA. Error bars=S.E.M.

### Example 8—Immunohistochemical Biomarkers of CBX2 Expression Correlate with Chemoresistance and Survival

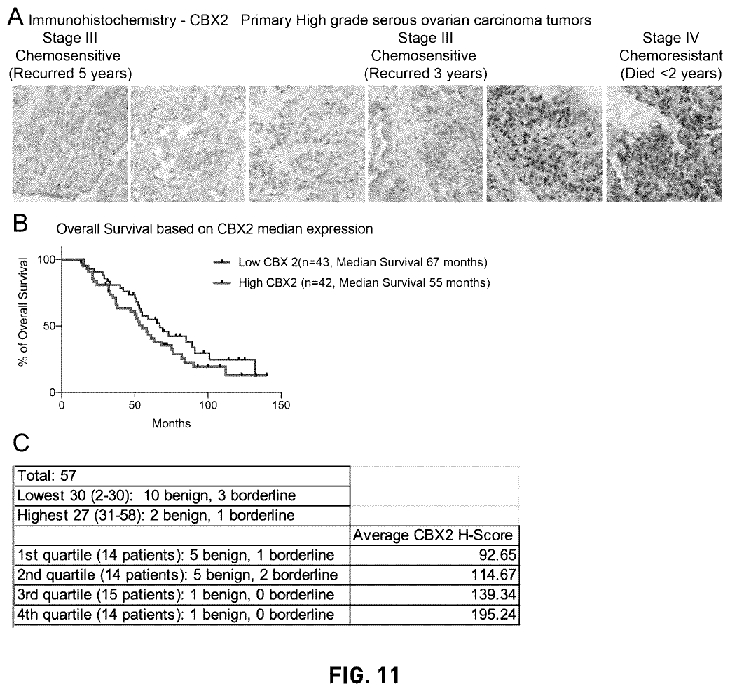

Using an optimized IHC protocol and a commercially available anti-CBX2 antibody, we examined CBX2 protein expression in 85 primary epithelial ovarian cancers (EOC) with varying degrees of chemosensitivity. A high CBX2 histology score correlated with more chemoresistant disease (FIG. 11 Panel A). However, while not statistically significant, elevated CBX2 protein portended a poorer overall survival, with the low-CBX2 expressing tumors having a 12-month survival benefit (FIG. 11 Panel B). Next, CBX2 protein expression was examined in a commercially available tissue microarray with a variety of benign (n=12), borderline (n=4), and malignant (n=41) gynecologic pathologies (FIG. 11 Panel C). Enrichment of low (1^{st}/2^{nd} quartile) CBX2 expressing tissues were benign or borderline (n=13) and only two benign tissues with high expressing CBX2 (3^{rd/}4^{th} quartile). Taken together, these data show that elevated CBX2 expression is associated with transformed tissue and correlates to therapy-resistant tumors.

### Example 9—Aurora Kinase A Inhibitor Alisertib Suppresses CBX2 Activity

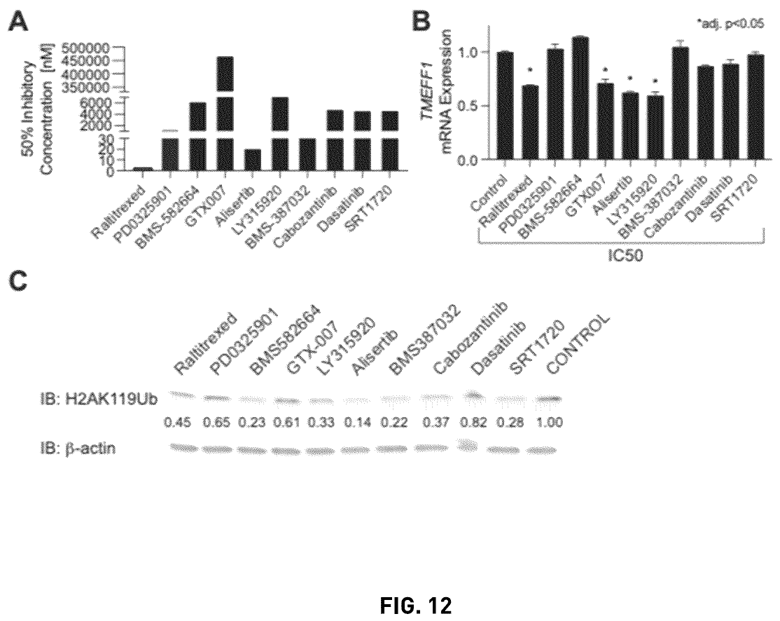

Ten candidate inhibitors were evaluated based on their ability to inhibit HGSOC cell viability. Dose response assays of all 10 inhibitors were conducted in OVCAR4, PEO1, and COV504 HGSOC cell lines to determine half inhibitory concentration (IC50) (FIG. 12 Panel A). We explored ability of these compounds to downregulate a CBX2 target gene, transmembrane protein with EGF-like and two follistatine-like domains 1 (TMEFF1) (FIG. 12 Panel B). TMEFF1 is a known CBX2 downstream gene, demonstrated to play a role in tissue development and tumor promotion. Four inhibitors, raltitrexed, GTX-007, LY315920, and alisertib, significantly inhibited TMEFF1 expression as measured by qPCR after treatment of OVCAR4 cells (FIG. 12 Panel B).

CBX2 is a known subunit of PRC1, which is critical for normal development and functions as a transcriptional repressor. PRC1 is understood to have histone ubiquitin ligase activity, specifically targeting Histone H2A Lys119 ubiquitination (H2AK119ub). In order to evaluate the impact of treatment with alisertib on the broader PRC1 complex, H2AK119ub was used as a surrogate marker of CBX2 activity in the PRC1. OVCAR4 (***confirm) cells were treated with IC50 doses of each of the 10 candidate inhibitors, a Western blot for H2AK119ub was compared to a beta-actin control (FIG. 12 Panel C). The results showed that alisertib decreased Histone H2A Lys119 ubiquitination, indicating inhibition of CBX2 activity in the PRC1.

### Example 10—Stemness as a Functional Component of CBX2 Inhibition

Loss of CBX2 has been demonstrated to attenuate the stem-like phenotype of HGSC and resensitize HGSOC to platinum. Stemness is also thought to play a role in the association between increased CBX2 and increased chemoresistance.

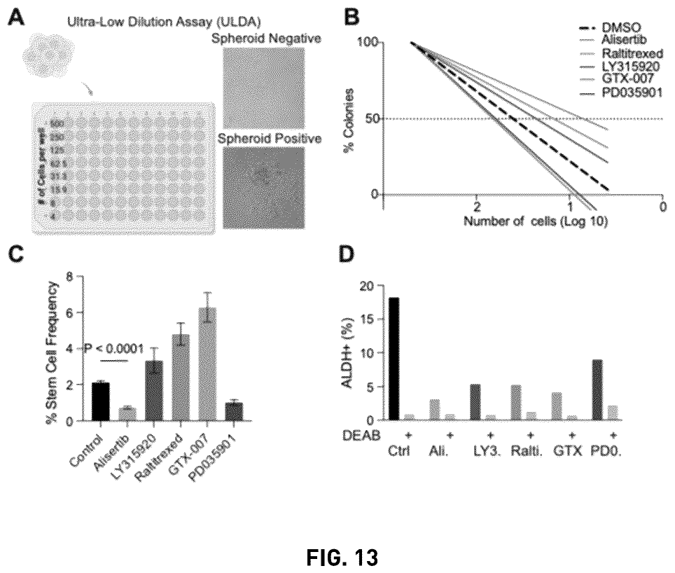

An ultralow dilutional assay (ULDA) utilizing the OVCAR4 HGSOC cell line was used to evaluate the functional impact of four inhibitors. The number of spheroids were plotted against the IC50 dose of each agent (FIG. 13 Panel B). The more significant the decrease in spheroids formation capacity, the more significant the decrease in stemness associated with treatment. This data was then quantified in graph form (FIG. 13 Panels B-C).

Finally, OVCAR4 cells were treated with the IC50 of each of the top 5 candidate inhibitors and the ALDEFLUOR™ assay was performed to identify the percentage of ALDH positive cells, compared to control (DEAB positive) (FIG. 13 Panel D).

Based on the understanding that CBX2 drives a stem-like phenotype, stemness assays were utilized to explore the functional implications of treatment with each of the top candidate compounds. These assays, across multiple cell lines, identified the Aurora kinase A inhibitor alisertib as the molecule which is most likely to interact with and inhibit CBX2.

### Example 11—Demonstration of Interaction and Dependence Between Alisertib and CBX2 and Polycomb Repressor Complex 1

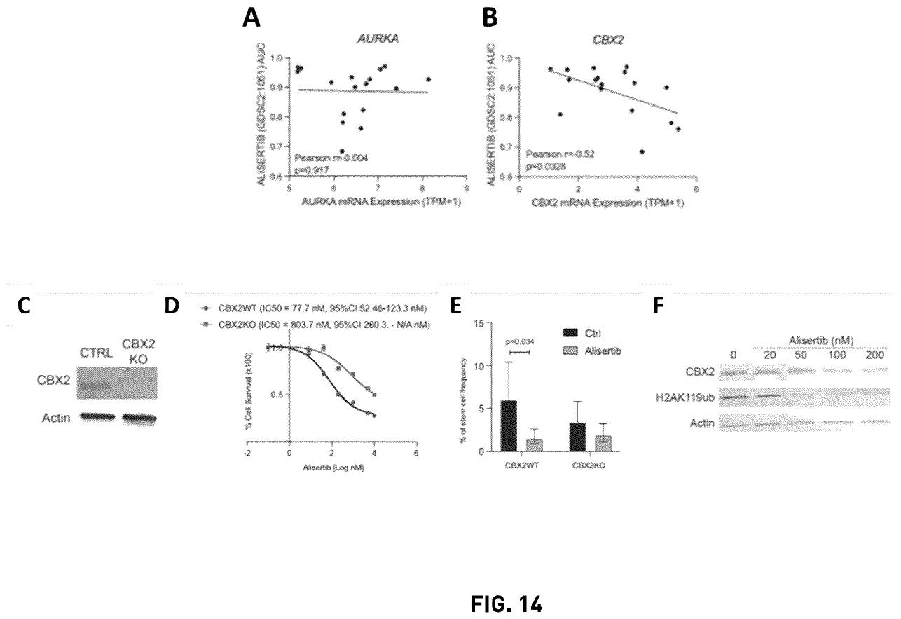

Understanding that the mechanism of alisertib is known to be inhibition of Aurora A kinase, The Cancer Dependency Map (CITE DepMap) was utilized to evaluate the expression of Aurora kinase A mRNA compared to expression of CBX2 mRNA based after treatment alisertib in all ovarian cancer cell lines. These values were calculated and R scores were calculated (FIG. 14 Panels A-B).

A CRISPR/Cas9 knock out of CBX2 was used to perform a proliferation assay of parental OVCAR4 cells with CBX2 intact compared to CBX2 knock out (CBX2 KO) and treated with increasing doses of alisertib (FIG. 14 Panels C-D). The IC50 of alisertib required to treat the CBX2 KO cells was ten times higher compared to the parental cells (FIG. 14 Panel D). A subsequent evaluation for stemness through ULDA showed that knocking out CBX2attenuated impact of alisertib on stemness (FIG. 14 Panel E). These findings suggest that the functional impact of alisertib in high grade serous cell lines is dependent on intact CBX2.

Finally, PRC1 target H2AK119ub was used to evaluate the impact of treatment with alisertib on the broader PRC1 complex. OVCAR4 and PEO1 cells were treated with increasing doses of aliserib and western blots were performed to evaluate H2AK119ub as a marker of PRC1 activity (FIG. 14 Panel F).

The results indicate a direct interaction between CBX2 and alisertib. The results further confirm the functional dependence of alisertib on intact CBX2, given attenuated alisertib effect in the setting of CRISPR CBX2 KO. Finally, a surrogate marker of broader PRC1 activity, H2AK119ub, was decreased in parallel with CBX2 after treatment with alisertib, reiterating the role of the PRC1 complex, and CBX2 specifically, in the function of alisertib.

### Example 12—Alisertib Inhibits Tumor Growth in a CBX2-Dependent Manner in Mouse Model

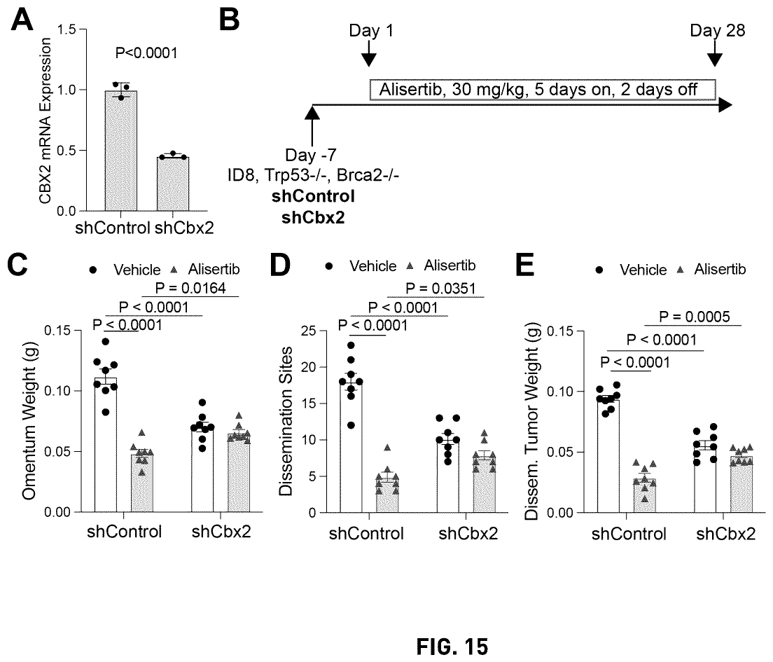

To further elucidate the dependence of alisertib on CBX2, an in vivo study was performed using a syngeneic mouse model with CBX2 intact compared to CBX2 knock down, undergoing treatment with alisertib. Measurements of mRNA expression confirmed that CBX2 knock down was effective (FIG. 15 Panel A). The mice were treated with vehicle control or 30 mg/kg alisertib for 28 days (FIG. 15 Panel B). Tumor-bearing mice were necropsied at the end of treatment and omental weight, number of dissemination sites, and total tumor weight were quantified (FIG. 15 Panels C-E, respectively). Each of these measurements showed a significant attenuation of the anti-tumor response of alisertib in CBX2 knock down mice as compared to that seen in CBX2 intact mice. It can be concluded therefore, that the loss of CBX2 expression in an ovarian cancer model attenuates the anti-tumor response to alisertib.

### Materials and methods

### 1. Cell Culture

OVCAR4, PEO1, OVCAR8 human high grade serous ovarian cancer cell lines and syngeneic murine cell line ID8 were authenticated using small tandem repeat (STR) analysis (The University of Arizona Genetics Core) and routinely tested for mycoplasma with MycoLookOut (Sigma, St. Louis, MO). OVCAR8 and OVCAR4 cells were obtained from the Gynecologic Tumor and Fluid Bank (University of Colorado, Aurora, CO). PEO1 purchased from American Type Culture Collection. The human cells were cultured in RPMI-1640 medium supplemented with 1% penicillin-streptomycin and 10% fetal bovine serum. ID8 cells were cultured in DMEM supplemented with 4% heat-inactivated FBS, 100 U/ml penicillin/streptomycin, 5 μg/ml insulin, 5 μg/ml transferrin and 5 ng/ml sodium selenite (Thermo Fisher, Massachusetts, USA). The cell lines were maintained in 5% CO2 at 37° C.

### 2. Bioinformatic Database Analysis

Gene Expression Omnibus (GEO), hosted by the National Center for Biotechnology Information (NCBI), was queried for relevant databases. GSE18521, GSE10971, GSE1926, and GSE73064 were examined for relative CBX2 expression. The Cancer Genome Atlas (TCGA) Ovarian Serous Cystadenocarcinoma (Nature 2011) database was accessed via the cBIOPortal (www.cbioportal.org/) to evaluate the role of CBX2 in HGSOC disease-free and overall survival. Note: patient numbers for overall survival (n=485) and disease-free survival (n=396) differ due to the availability of data with TCGA. The database was queried for HGSOC identifying a total of 557 tumors with mRNA expression data and CBX2 upregulation was defined as CBX2 mRNA expression >1.5 standard deviation. RPPA data was assessed from the HGSOC Provisional dataset via the cBIOPortal.

### 3. Adherent and Suspension Environments

The adherent environment was created using standard tissue culture dishes. For suspension, tissue culture dishes were covered with 6 mg/ml poly-2-hydroxyethyl methacrylate (Poly-HEMA, Sigma) in 95% ethanol. The plates were incubated under sterile conditions to allow ethanol evaporation, followed by 30 min of ultraviolet light for sterilization. OVCAR4, PEO1, and OVCAR8 cells were cultured in each of these environments for 7 days.

### 4. Cisplatin Dose-Response

PEO1 and OVCAR4 cells were plated in 96-well plates in both adherent (4000 cells per well) and suspension (6000 cells per well) environments, as described above. These cells were treated over 24 h with increasing concentrations of Cisplatin (0.5-100 μM). 1640 RPMI media and media with 0.9% NaCl were used for control and vehicle control, respectively. Cell viability was assessed using the 3-(4,5-dimethylthiazol-2-yl)-5-(3-carboxymethoxyphenyl)-2-(4-sulfophenyl)-2H-tetrazolium (MTT) assay (Promega, Madison, WI). Means of at least five wells are reported and experiments were independently repeated in triplicate. Representative dose-response curves are shown.

### 5. Quantitative Reverse Transcription Polymerase Chain Reaction (RT-qPCR)

RNA was isolated using RNAeasy® Plus Mini Kit (Qiagen, Hilden, Germany) according to the manufacturer's protocol. NanoDrop® spectrophotometry was performed to confirm the concentration of extracted RNA. RT-qPCR was performed using the Luna® Universal One-step RT-qPCR kit (New England BioLabs, Ipswich, MA) on a BioRad® CFX96·8 or Applied Biosystems QuantStudio™ 6 Flex thermocycler using primers for specific target transcripts; 18s rRNA was examined as a housekeeping gene (Table 5).

TABLE 5

| Gene | Direction | Sequence (5′-3′) | SEQ ID NO: |
|---|---|---|---|
| CBX2 | Forward | CGGCTGGTCCTCCAAACATAA | 1 |
| CBX2 | Reverse | CAGAACCGGAAGAGAGGCAA | 2 |
| 18sRNA | Forward | AACTTTCGATGGTAGTCGCCG | 3 |
| 18sRNA | Reverse | CCTTGGATGTGGTAGCCGTTT | 4 |
| ALDH3B1 | Forward | TACGCCTTCTCCAACAGCAG | 5 |
| ALDH3B1 | Reverse | GTCATGTGCATGAAGCCGTC | 6 |
| ALDH2 | Forward | GGTGTGGTCAACATTGTGCC | 7 |
| ALDH2 | Reverse | ATTACGCGGCCAATCTCAGT | 8 |
| ALDH3A1 | Forward | AAGAGGAGATCTTCGGGCCT | 9 |
| ALDH3A1 | Reverse | TAATCACCTTGTCGTTGCTGGA | 10 |
| MYLK | Forward | TCAACAGGGTCACCAACCAG | 11 |
| MYLK | Reverse | GGGGTCTGGGTATCCTTCAAT | 12 |
| NOG | Forward | TGGTGGACCTCATCGAACAC | 13 |
| NOG | Reverse | ATGAAGCCTGGGTCGTAGTG | 14 |
| TNFSF10 | Forward | TGCGTGCTGATCGTGATCTT | 15 |
| TNFSF10 | Reverse | CATTCTTGGAGTCTTTCTAACGAGC | 16 |
| SOX4 | Forward | CCCAGCAAGAAGGCGAGTTA | 17 |
| SOX4 | Reverse | CATCGGCCAAATTCGTCACC | 18 |

### 6. shRNA Knockdown

Examples 1-7: CBX2-specific shRNA were obtained from the University of Colorado Functional Genomics Facility (CBX2 #1: TRCN 0000020327 and CBX2 #2: TRCN 0000020328). An empty pLKO.1-puro was utilized as shControl (shCtrl). Plasmid isolation was performed using Plasmid Midi-Prep Kit (Qiagen). Twenty-four hours after seeding, cells were transfected with a total of 12 μg of DNA, including lentiviral packaging plasmids and the shRNA, in addition to 36 μg of polyethyenimine (PEI), for a 1:3 ratio of DNA to PEI. Cells were incubated overnight and transitioned to Dulbecco's Modified Eagle Media (DMEM) the following morning. Forty-eight hours after medium change, lentivirus was harvested. PEO1, OVCAR4, and OVCAR8 cells were seeded into six-well plates. When cells reached 80% confluence, they were transduced with lentivirus encoding CBX2-specific shRNAs or an shRNA control. A control well was maintained without virus to confirm puromycin selection. A 48-h puromycin selection was performed immediately following transduction. After medium change, cells were allowed to recover and then subjected to functional assays.

Examples 8-12: CBX2-specific shRNAs were obtained from the University of Colorado Functional Genomics Facility (human CBX2 #1: TRCN 0000020327 and human CBX2 #2: TRCN 0000232722). For Mouse shCbx2, shRNA sequence of the TRCN0000334429 was cloned into pLKO.1-blast vector (Addgene, 26655) using EcoRI and AgeI site. An empty pLKO.1-puro and pLKO.1-blast was utilized as shControl (shCTRL). Plasmid isolation was performed using Plasmid Midi-Prep Kit (Qiagen Hilden, Germany). HEK293T cells were transfected with lentivirus construct with packaging plasmids with Lipofectamine 2000 (Thermo Fisher Scientific, Waltham, MA) following manufacture's instruction. Cells were incubated overnight and transitioned to Dulbecco's Modified Eagle Media (DMEM) the following morning. Viral supernatant was collected 72 hours post-transfection, applied to OVCAR4, PEO1 or ID8 Trp53^{-/-} Brca1^{-/-} for 24 hours with Polybrene. Infected cells were selected using 1.0 ug/ml puromycin.

### 7. Immunoblot

Cell lysis was performed using radioimmunoprecipitation assay (RIPA) buffer (150 mM sodium chloride, Triton X-100, 0.5% sodium deoxycholate, 0.1% SDS [sodium dodecyl sulfate], 50 mM Tris, pH 8.0) supplemented with complete EDTA-free protease inhibitor cocktail (Roche), as well as NaF and NaV. Protein was quantified using bicinchoninic acid (BCA) protein assay (Thermo Fisher Scientific, Waltham, MA) and spectrophotometry. An 8% SDS polyacrylamide gel resolving gel was created with a 4% stacking gel. Twenty to thirty micrograms of total protein were loaded per well. Proteins were transferred to PVDF membrane using a Bio-Rad® TransBlot® Turbo™. Western Blot analysis was performed using a rabbit primary antibody specific to CBX2 (Thermo Fisher Scientific, Cat #PA5-30996, 1:1000), a mouse primary antibody against actin (Abcam, Cat #ab6276) and anti-H2AK119Ub (Cell signaling Cat #8240). CBX2 previously validated in Clermont, P. L. et al. Genotranscriptomic meta-analysis of the Polycomb gene CBX2 in human cancers: initial evidence of an oncogenic role. Br. J. Cancer 111, 1663-1672 (2014). Primary antibody incubation was performed overnight at 4° C. Secondary goat anti-rabbit green (LI-COR Biosciences, Lincoln, NE, Cat #926-32211) and goat anti-mouse red (LI-COR, Cat #926-68070) antibodies were applied the following morning for 1 h at room temperature. Bands were visualized using the LI-COR Odyssey® Imaging System.

### 8. Proliferation Assays

Examples 1-7: For the Gaussia luciferase (gLuc) assay, the BioLux® Gaussia Luciferase Assay kit (New England BioLabs) was utilized. OVCAR4 and PEO1 cells were grown in a 96-well plate, starting with 2000 cells per well. Media were collected every 24 h and stored in at −20°. For the assay and luminometer readings, the media were thawed and placed in a new 96-well plate. The assay was performed following the manufacturer's protocol and the relative light units were obtained using luminometry (GloMax) and charted with Prism software. Colony formation assays were performed in parallel using crystal violet staining. Briefly, cells were fixed (10% methanol/10% acetic acid/PBS), stained with crystal violet (0.4%) and washed with de-ionized water. Crystal violet was dissolved and absorbance was measured using a spectrophotometer (SpectraMax® M2e, Molecular Devices, San Jose, CA) at 590 nm and SoftMax® Pro software. For the spheroid assay, 4000 cells were plated from a single cell suspension onto growth factor reduced Matrigel (Corning, Corning, NY) and allowed to incubate for 12 days. Microscopic images were obtained and the diameter of each spheroid was measured in ImageJ (NIH). At least 50 spheroids were measured for each cell type and the diameters were averaged and graphed using Prism software.

Examples 8-12: All drugs were suspended in DMSO at the concentration of 50 mM and aliquoted and stored in −80 C. 1000 cells were seeded in 96 well plate a day prior. 10 fold dilution of drugs in the cultureing media supplemented with FBS and penicillin/Streptomycin was added and cells were cultured until the cells in the no-drug added wells became confluent (5-8 days). Cells were washed with PBS twice and fixed with fixation buffer (10% Methanol and 10% Acetic acid in PBS) followed by staining with Cristal Violet staining solution (0.4% Cristal Violet in 20% Ethanol). After Cristal violet in the wells were washed away with distilled water, plates were dried overnight, Cristal violet on the cells were dissolved in fixation buffer and quantified by plate reader (OD570).

### 9. Aldefluor™ Assays

Stemness was evaluated using the Aldefluor™ Kit (Cat #01700, Stemcell Technologies, Vancouver, Canada).

Examples 1-7: OVCAR4 and OVCAR8 cells grown in adherent and suspension settings for 7 days were collected and prepared following manufacturer's protocol. An exception to the manufacturer's protocol was the reduction of Aldefluor™ reagent by 50%, using 2.5 μl per 1 ml of cell suspension. Flow cytometry was performed on a Gallios® 561 Cytometer (BD Biosciences) with analysis at 488 nm.

Examples 8-12: Briefly, 100,000 cells were trypsinized and resuspended in ALDEFLUOR buffer or buffer with diethylaminobenzaldehyde (DEAB) as negative control. ALDEFLUOR reagent was added and incubated at 37° C. for 45 minutes. Cells were washed with cold HBSS buffer (2% FBS HBSS) twice. Cells were stained with Zombie Red (BioLegend 423109) for viavility. ALDH activity was analyzed using Novocyte Penteon (Agilent). Cytometry data were analyzed using FlowJo™ software (Tree Star).

### 10. Gynecologic Tissue and Fluid Bank (GTFB)

The University of Colorado has an Institutional Review Board approved protocol (COMIRB #07-935) in place to collect tissue from gynecologic patients with both malignant and benign disease processes. All participants are counseled regarding the potential uses of their tissue and sign a consent form approved by the Colorado Multiple Institutional Review Board. The tissues are processed, aliquoted, and stored at −80° C.

### 11. Immunohistochemistry

Patient-derived xenograft tissue samples of HGSOC were fixed in formalin and embedded in paraffin. The University of Colorado Cancer Center Histology Core performed serial sectioning of the tissue at 5 micron thickness. For histopathologic examination, sections were de-paraffinized using xylene and hydrated in graded alcohol solutions. Antigen retrieval was performed using citrate buffer (pH 6.0) and boiling in pressurized steamer to 110° C. for 30 min. Endogenous peroxidase activity was quenched with 3% hydrogen peroxide in methanol for 20 min, followed by washing in TBS. A hydrophobic barrier was drawn around each section and tissues were blocked in 1% BSA in TBS for 30 min. TMA slides were single stained. Rabbit anti-CBX2 (Thermo Scientific, Cat #PA5-30996) was diluted to 1:50 in 1% BSA in TBS, applied to all sections, and incubated overnight at 4° C. Rabbit anti-PAX8 (Proteintech, Cat #10336-1-AP) was diluted to 1:200 in 1% BSA in TBS, applied to all sections, and incubated overnight at 4° C. An isotype control (Rabbit IgG) was incubated in parallel. The secondary antibody, anti-rabbit Dako Envision™+System HRP Labeled Polymer (Dako Ref #K4003) was applied to the sections and allowed to incubate for 60 min at room temperature. Slides were subsequently washed with TBS and developed under the microscope using Liquid 3,3′-diaminobenzidine tetrahydrochloride (DAB)+Substrate Chromagen System (Agilent, Santa Clara, CA; Ref #K3468). Slides were counterstained with hematoxylin.

### 12. Tissue Microarray

A previously constructed TMA comprised of matched primary, lymph node, and peritoneal metastases samples in duplicate from 24 patients with high grade serous carcinoma treated at the University of Colorado (COMIRB #14-0427), was stained with the CBX2 and PAX8 antibodies. With the aid of the University of Colorado Histology Core, the stained slides were scanned using Aperio imaging technology and annotated to highlight tumor based on PAX8 staining using ImageScope software. The TMA was then analyzed and scored by the University of Colorado Histology Core. Subsequently, two board-certified pathologists (M.D.P. and A.A.B.) manually reviewed and scored the CBX2 stain. Each sample was given a score for intensity (0, 1+, 2+, 3+) and percentage of cells staining (continuous variable). Only distinct nuclear staining of tumor cells was considered positive. From this data, H-scores were generated.

### 13. Apoptosis Assays

Following 72 h of growth in adherent and suspended states, OVCAR4 and PEO1 cells were harvested and washed in PBS. Alexa Fluor™ 488 Conjugated AnnexinV and Propidium Iodide (PI) (Thermo Fisher Scientific) staining were performed following manufacturer's protocol.

### 14. Quantitative Reverse-Transcriptase PCR (qPCR)

RNA was isolated using RNAeasy Plus Mini Kit (Qiagen, Hilden Germany) according to the manufacturer's protocol. NanoDrop spectrophotometry was used to measure concentration of extracted RNA. Luna Universal One-step RT qPCR kit (New England BioLabs, Ipswich, MA) was used on BioRad CFX96 (Bio-Rad, Hercules, CA). HPRT or GAPDH were used as internal control as stated in figure legend.

### 15. CRISPR Knock Out of CBX2

CBX2 CRISPR knocked-out OVCAR4 cells were created by the University of Colorado Functional Genomics Facility using the IDT Alt-R RNP system. ALT-R crRNA and ATL-R tracrRNA were suspended at 100 μmol/L in nuclease-free IDTE pH7.5. The same volumes of ALT-R crRNA and ATL-R tracrRNA were mixed to prepare gRNA complex at 50μmol/L, heated at 95° C. for 5 minutes, and then were cooled to room temperature. The RNP complex was prepared with 150 pmol of gRNA and 125 pmol of Alt-R Cas9-NLS in final volume of 5 μL in PBS. Cells were harvested in Necleofector solution SF with supplement (Lonza, Basel, Switzerland) at a concentration of 1×106 cells per 100 μL. The transfection mix was made with 100 μL of cell suspension, 2.5 μL each of RNP complex, and 0.6 μL of 100 μL of Alt-R Cas9 Electroporation enhancer. For OVCAR4, program FE-132 was used. After pulsing, prewarmed medium was added into the Nucleocuvette vessel, and cells were plated for further culturing and isolating clonal populations. The following guide RNAs were used: Hs.Cas9.CBX2.1.AC: CCGAGTGCATCCTGAGCAAG (SEQ ID NO. 21) and Hs.Cas9.CBX2.1.AA: GAGTACCTGGTCAAGTGGCG (SEQ ID NO. 22). PCR primers to screen for deletion included CBX2-F1: AGCATGGAGGAGCTGAGCA (SEQ ID NO. 23), CBX2-R2: GGTTACAGCGGGGAGAATCTG (SEQ ID NO. 24), and CBX2-R3: GGAGAATCTGGCCAAGAGGAG (SEQ ID NO. 25).

### 16. Ultra Low Dilution Analysis (ULDA) and MTT Assay

Cells were trypsinized and filtered with a 40-micron filter, creating a single cell suspension. Cells were plated in 96 wells ultra-low attachment plates (CORNING #3474 this is flat bottom, and CORNING #7007: used u bottom for COV504) in 200 ul CSC media (DMEM/F12, 1× B27, 4% FBS, 100 U/ml penicillin/streptomycin, 20 ng/ml human EGF, 20 ng/ml human FGF). After 3 days, 100 μl of medium was gently taken out, then added drugs to final IC50 concentration. The media with drug was replaced with half volume every 4-5 days and cultured for 3 weeks. After 3 weeks of culture, MTT was added (3-(4,5-dimethylthiazol-2-yl)-2,5-diphenyltetrazolium bromide) to a final concentration of 500 μg/ml. Plates were incubated at 37° C. for 4 hours. After 4 hours spheroid existence and color were evaluated under the microscope (Incucyte). Observation of purple spheroids confirmed the presence of live cells and were scored as 1. If no spheroids appreciated, or if purple spheroids were not observed it was scored as 0. Plotted on the graph. The stem cell frequency was calculated using ultra low limiting dilution analysis (PMID: 19567251).

### 17. Mouse In Vivo Model

All mouse experiments were approved by the Institutional Animal Care and Use Committee (IACUC protocol #569). Six-to eight-week-old C57BL/6J mice were purchased from The Jackson Laboratory (strain #000664), and each mouse was intraperitoneally (i.p.) injected with 5×10⁶ ID8 Trp53^{-/-} Brca2^{-/-} cells, followed by a seven-day tumor establishment period prior to the initiation of drug treatment. In the second study, the cells were either CBX2-intact or CBX2-depleted. Briefly, CBX2-intact cells were treated with an empty lentiviral vector (shControl) or a lentiviral vector containing CBX2-specific small hairpin RNA (shCbx2), as described above. Body weights were collected twice per week and served as a surrogate for drug toxicity. Mice were euthanized the day after completion of the 28-day drug treatment regimens, and tumor burden was assessed through omentum weight, number of disseminated tumor sites, and total disseminated tumor weight. Tissue was also collected and processed for further analysis. In the first study, blood was collected via submandibular puncture prior to death, and complete blood cell (CBC), aspartate transaminase (AST), and alanine aminotransferase (ALT) levels were assessed for signs of drug toxicity.

### Drug Treatment Regimens

In the first study, mice were treated with daily 30 mg/kg alisertib (MCE, Cat. #HY-10971/CS-0106) via oral gavage (PO) and/or weekly 1 mg/kg cisplatin (Selleckchem, Cat. #S1166) via i.p. injection for 28 days. Control mice were treated with vehicle (daily 5% DMSO, 35% PEG300, 5% Tween80 in sterile H₂O, PO, and weekly sterile PBS, i.p.). Importantly, the alisertib-treated mice began to lose weight near the end of the study, so they were given a three-day alisertib “vacation” on days 23-25 of treatment.

In the second study, mice were treated with 30 mg/kg alisertib, PO, for 28 days, as described above. Due to the weight loss observed in the first study, we reduced treatment from 7 days per week to 5 days per week.

### 18. Statistical Analysis

Examples 1-7: Prism GraphPad software (v7) was utilized to generate graphs. Statistical tests include unpaired two-sided t-tests (comparing two groups), Log-rank (survival) or ANOVA (comparing greater than two groups) unless noted. A significance threshold was set at p<0.05, which was used for sample size determination. All experiments were performed in technical triplicates and biological triplicates unless noted. FloJo software (BD Biosciences, San Jose, CA) was used for analyzing flow cytometry data.

Examples 8-12: All statistical analysis was conducted in Prism GraphPad v10. Survival comparison used Kaplan-Meier with Logrank, pairwise comparison used t-test, multicomparison used Analysis of Variance with multiple test correction, pairwise comparison over time was analyzed using a mixed model effect. A p<0.05 was considered significant and when required False Discovery Rate multicomparison correction (q<0.05) was made. Error bars are shown as standard error mean (SEM). All in vitro experiments were performed in triplicate, and the in vivo experiment was run in duplicate.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A method of classifying a tumor for treatment, comprising:
obtaining a sample of a tumor;
processing the sample to determine a measurement of Chromobox 2 (CBX2) expression in the tumor;
assigning a score to the measurement; and
classifying the tumor based on anticipated responsiveness to a treatment with an Aurora kinase A inhibitor, wherein classifying comprises grading the score based upon a scale that includes a minimum score that identifies the tumor as likely responsive to the treatment.

2. The method of claim 1, wherein the measurement is an amount of CBX2 protein in the sample.

3. The method of claim 1, wherein processing comprises performing an immunohistochemistry (IHC) assay on the sample, and wherein the score comprises one or more of an H-score, Allred score, or immunoreactive score.

4. The method of claim 3, wherein the score comprises an H-score, and the minimum score is about 90 to about 300.

5. The method of claim 4, wherein the minimum score is at least about 120.

6. The method of claim 4, wherein the minimum score is at least about 150.

7. The method of claim 4, wherein the minimum score is at least two times greater than an H-score for an autologous non-tumor tissue sample.

8. The method of claim 4, wherein the minimum score is at least three times greater than an H-score for an autologous non-tumor tissue sample.

9. The method of claim 1, wherein the Aurora kinase A inhibitor is alisertib.

10. A method of determining a treatment for a patient with an ovarian cancer, the method comprising:
obtaining a first sample of a tissue of Müllerian origin from the patient comprising one or more cancerous or pre-cancerous cells;
obtaining a second sample of a tissue of Müllerian origin from the patient but does not comprise cancerous or pre-cancerous cells;
processing the first sample and the second sample to analyze at least one biomarker of Chromobox 2 (CBX2) expression, wherein the at least one biomarker comprises one of: a CBX2 mRNA sequence, CBX2 protein or a fragment thereof, aldehyde dehydrogenase, and SOX4; and
quantifying the amount of the at least one biomarker in the first sample and the second sample, wherein if the amount of the at least one biomarker in the first sample is greater than the amount of the at least one biomarker in the second sample, the patient is identified as having a cancer that is likely responsive to a treatment with an Aurora kinase inhibitor.

11. The method of claim 10, wherein the at least one biomarker is CBX2 protein or a fragment thereof, and wherein the quantifying step comprises performing an immunohistochemistry (IHC) assay on the first sample and the second sample.

12. The method of claim 11, further comprising assigning a score to each of the first sample and the second sample, wherein the score comprises one or more of an H-score, Allred score, and an immunoreactive score.

13. The method of claim 12, wherein the score comprises an H-score, and wherein the patient is identified as having a cancer that is likely responsive to a treatment with an Aurora kinase inhibitor if the H-score for the first sample is at least two times greater than the H-score for the second sample.

14. The method of claim 13, wherein the patient is so identified if the H-score for the first sample is at least three times greater than the H-score for the second sample.

15. The method of claim 10, further comprising administering a therapeutically effective amount of the Aurora kinase A inhibitor to the patient.

16. The method of claim 10, wherein the Aurora kinase A inhibitor is alisertib.

17. A kit for classifying a tumor for treatment, comprising an article providing a scale of immunohistochemistry scores, and that indicates a minimum score that identifies a sample having that score as coming from a tumor that is likely responsive to a treatment with an Aurora kinase inhibitor.

18. The kit of claim 17, wherein the article is a printed material.

19. The kit of claim 17, wherein the scale comprises one or more of H-scores, Allred scores, and immunoreactive scores.

20. The kit of claim 17, wherein the scale comprises H-scores, and the minimum score is about 90 to about 300.
